(19) United States
(12) Patent Application Publication
(10) Pub. No.: US 2004/0136905 A1
(43) Pub. Date: Jul. 15, 2004

(54) Magnetically guided particles for radiative therapies

(76) Inventors: Thomas B. Kent, Boulder, CO (US); Jacqueline Johnson, Rancho Santa Fe, CA (US); Scott R. Rudge, Boulder, CO (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/687,555

(22) Filed: Oct. 15, 2003

Related U.S. Application Data
(60) Provisional application No. 60/419,228, filed on Oct. 15, 2002.

(51) Int. Cl. A61K 51/00; A61K 33/26

(52) U.S. Cl. 424/1.11; 600/1; 424/646

(57) ABSTRACT

Certain magnetic compositions may be used successfully for injection, followed by the application of an externally placed magnetic field that guides the particles to the targeted site, such as tissues, cells or cell components. The particles are extravasated into the targeted tissue, affording better localized distribution of the particles throughout the targeted tissue and to deeper target tissue sites. The relatively small size of the particles yields a more uniform radiative therapy, while utilizing smaller total particle mass for the procedure.

### CROSS REFERENCE TO RELATED APPLICATIONS

This application claims priority to Provisional Application No. 60/419,228 entitled MAGNETICALLY GUIDED PARTICLES FOR RADIATIVE THERAPIES, filed Oct. 15, 2002. The subject matter of the aforementioned application is hereby incorporated herein by reference in its entirety.

### BACKGROUND OF THE INVENTION

The effectiveness of radiative therapy as a treatment modality for tissue destruction is known. For example, seed implantation, followed by hyperthermia therapy has shown limited success in treating tumors. This technique has been named interstitial implant hyperthermia (IIH). There are shortcomings of this type of therapy, however, due in part to the poor distribution of particles throughout the target tissue that is afforded by the manual placement of the relatively large seed particles, which leads to nonuniform heating and ineffective tissue destruction. To address this, increasing amounts of energy must be applied, leading in many cases to damage or destruction of adjacent healthy tissues that are not in need of treatment. Additionally, if there is high blood flow in the area of the seed, the blood may cool the temperature of the seed and thus reduce the effectiveness of the treatment. Likewise, since surgery is usually required for the implantation of the seed, there is a risk of infection.

An alternative to the placement of large seeds is arterial embolization hyperthermia (AEH). AEH involves the use of the arterial blood supply of the tumor to provide access for particles. Following the injection of the particles into the blood supply of the tumor, and hopeful association of the particles with the tumor, an alternating magnetic field can be used to heat the particles and attempt to damage the local tissue. However, this technique is limited to tumors with good blood supplies. Likewise, very small disease foci, which have no independent blood supply, are not amenable to this technique. Additionally, organs with only a single arterial supply run a very high risk of having that single arterial supply damaged by the heating step. Thus, embolization techniques are limited almost exclusively to the liver.

Another alternative is direct injection hyperthermia (DIH). Once again, magnetic particles are administered to the patient, however, this time, the particles are added to a carrier fluid so that they can be injected directly into the tumor prior to the application of an alternating magnetic field. This technique does not allow for even or complete distribution of the particles throughout the targeted site. This is a major limitation since the effectiveness of the therapy is related to the lowest energy dose delivered to any portion of the tumor, while the safety is related to the highest energy dose delivered to surrounding healthy tissue. Additionally, since the particles are administered at a point location, visualization of the tumor is key in having an effective treatment. Additionally, the need for injection into the tumor increases the risk of the tumor spreading.

Another alternative is intracellular hyperthermia (IH). This technique involves very small sized particles, small enough to be taken up by local cells. The particles are also coated with some material that allows their intracellular uptake. Again, as above, following the particle's positioning in the cells, an alternating magnetic field is applied to the cells to cause an increase in heat in the particles. A major problem with this technique is that a large number of the particles need to be taken up by each of the cells in order for the applied field to be able to induce enough heat in the cells to cause cell death. If sufficient amounts are not taken up, then, as above, not all of the tumor cells will be killed. Likewise, a major limitation is that the current state of technology only allows the delivery of such particles to a tumor either via a direct injection, or via a blood supply.

Other more general radiative techniques suffer from similar limitations. For example, external beam radiation therapy must irradiate intervening tissues, as well as tissue anterior to the targeted site, in order to deliver an adequate dose to the disease site. Thus, the effect of radiated energy on any disease site is limited by the least amount of energy delivered to any section of that site, while the harmful effects of the same radiation to adjacent tissues is related to the maximum energy deposited in those tissues. There remains a need for methods to increase the deposition of the desired energy in the targeted site, while limiting such deposition in healthy adjacent sites.

### SUMMARY OF THE INVENTION

Some aspects of the present invention are described in the following paragraphs:

1. A method of radiative therapy comprising:

a) introducing more than one magnetic component particle into a patient;

b) magnetically guiding with a non-alternating magnetic field the magnetic component particle to a targeted site; and

c) depositing energy at the targeted site;

2. The method of paragraph 1, wherein the magnetic component particle comprises a metal with more than 75% metallic iron;

3. The method of paragraph 1, wherein iron in the magnetic component particle is less than 10% iron oxide;

4. The method of paragraph 1, wherein the magnetic component particles comprises a magnetosorptive particle;

5. The method of paragraph 4, wherein the magnetosorptive particle has a weight ratio of magnetic component:sorbent in the range from about 95:5 to about 50:50;

6. The method of paragraph 4, wherein the magnetosorptive composition comprises magnetocarbon particles;

7. The method of paragraph 6, wherein the magnetocarbon particles comprise at least one type of activated carbon, selected from the group consisting of type A, type B, type E, type K, and type KB;

8. The method of paragraph 6, wherein the magnetocarbon particles further comprise one or more biologically active agents;

9. The method of paragraph 8, wherein the one or more biologically active agents are selected from the group consisting of antibiotics, antifungals and antineoplastic agents;

10. The method of paragraph 4, wherein the magnetosorptive composition comprises magnetoceramic particles;

11. The method of paragraph 10, wherein the ceramic is selected from the group consisting of a natural porous adsorptive material and a synthetic porous adsorptive material;

12. The method of paragraph 10, wherein the ceramic is selected from the group consisting of hydroxyapatite, silicas and chemically modified silicas;

13. The method of paragraph 10, wherein the magnetoceramic particles further comprise one or more biologically active agents;

14. The method of paragraph 13, wherein the one or more biologically active agents are chosen from the group consisting of antifungals, antineoplastics and antibiotics;

15. The method of paragraph 1, wherein the magnetic component particles are magnetopolymer particles;

16. The method of paragraph 15, wherein the polymeric components are biodegradable polymers.

17. The method of paragraph 16, wherein the polymeric component is PLGA;

18. The method of paragraph 15, wherein the magnetopolymer particles further comprise one or more biologically active agents;

19. The method of paragraph 15, wherein the one or more biologically active agents are chosen from the group consisting of antifungal, antineoplasic and antibiotics.

20. The method of paragraph 1, wherein the magnetic component particles are processed;

21. The method of paragraph 20, wherein the process is selected from the group consisting of gas phase treatment, mechanical milling, spray drying, heating, cooling, annealing, and plastic deformation;

22. The method of paragraph 1, where the magnetic component particles further comprise one or more biologically active agents that are one or more isotopes;

23. The method of paragraph 1, wherein one or more biologically active bifunctional agent are attached to the particles;

24. The method of paragraph 1, wherein the size of the particles is less than 5 cm;

25. The method of paragraph 24, wherein the average size of the particles in the magnetic composition is between approximately 0.1 microns to approximately 20 microns;

26. The method of paragraph 24, wherein the average size of the particle is from between about 0.5 to about 5 microns;

27. The method of paragraph 1, wherein the magnetic component particles are introduced with a delivery vehicle;

28. The method of paragraph 1, wherein the magnetic component particles are introduced with one or more excipients;

29. The method of paragraph 1, wherein the particles are introduced by a method selected from the group consisting of injection, infusion, implantation, and ingestion;

30. The method of paragraph 1, wherein the targeted site is selected from the group consisting of tumors, infections, aneurysms, abscesses, viral growths, and other focal points of disease;

31. The method of paragraph 1, also comprising the introduction of an embolic agent;

32. The method of paragraph 32, wherein the embolic agent is a second batch of magnetic component particles, wherein the larger particles are used as the embolic agent;

33. The method of paragraph 1, wherein the deposited energy is applied for an amount of time effective to obtain a therapeutic effect;

34. The method of paragraph 1, wherein protective compositions are used in the area surrounding the target;

35. The method of paragraph 1, wherein the deposited energy is applied with a RF capacitive heating system;

36. The method of paragraph 1, wherein the deposited energy is tunable;

37. The method of paragraph 1, wherein the deposited energy is electrical;

38. The method of paragraph 1, wherein the deposited energy is alternating magnetic energy;

39. The method of paragraph 1, wherein the deposited energy is nuclear;

40. The method of paragraph 39, wherein the nuclear energy is from gamma particles;

41. The method of paragraph 39, wherein the nuclear energy is from beta particles;

42. The method of paragraph 39, wherein the nuclear energy is from alpha particles;

43. The method of paragraph 39, wherein the nuclear energy is from neutrons;

44. The method of paragraph 43, wherein the neutrons are used for neutron capture therapy;

45. The method of paragraph 39, wherein the deposited energy is from heavy particles;

46. The method of paragraph 39, wherein the deposited energy is from a particle beam;

47. The method of paragraph 1, wherein the deposited energy is absorbed by the magnetic component particles and causes the release of one or more biologically active agents from the particles;

48. The method of paragraph 1, wherein the deposited energy is photon related;

49. The method of paragraph 1, wherein the deposited energy causes a beneficial rise or fall in local temperature;

50. The method of paragraph 1, wherein the deposited energy is ultrasound;

51. The method of paragraph 1, wherein magnetic component particles further comprises a biologically active agent;

52. A kit for administering radiative therapy, comprising:

a) a unit dose of magnetic component particles;

b) a non-alternating magnet for guiding said particles to a target in the patient once administered to the patient;

c) a source of energy that will deposit energy into the patient once the magnetic component particles have been administered to the patient and magnetically guided to the target;

d) optionally one or more receptacles and instructions for use;

53. The kit of paragraph 52, wherein the magnetic component particles comprises less than 10% iron oxide;

54. The kit of paragraph 52, wherein the magnetic component particles comprise a metal with more than 75% metallic iron;

55. The kit of paragraph 52, wherein the magnetic component particles comprise magnetocarbon particles;

56. The kit of paragraph 52, wherein the magnetic component particles comprise magnetoceramic particles;

57. The kit of paragraph 52, wherein the magnetic component particles comprise magneto-polymer magnetic component particles;

58. The kit of paragraph 52, wherein the magnetic component particles further comprise one or more biologically active agents;

59. The kit of paragraph 58, wherein the one or more biologically active agents are chosen from the group consisting of antifungals, antineoplastics and antibiotics;

60. The kit of paragraph 52, also comprising an embolic agent;

61. The kit of paragraph 52, wherein the source of energy is a RF capacitive heating system;

62. The kit of paragraph 52, wherein the source of energy is tunable;

63. The kit of paragraph 52, wherein the source of energy is a source of neutrons;

64. The kit of paragraph 52, wherein the source of energy is a source of gamma rays;

65. The kit of paragraph 52, wherein the source of energy is a source of beta particles;

66. The kit of paragraph 52, wherein the source of energy is a source of alpha particles;

67. The kit of paragraph 52, wherein the source of energy is a source of heavy particles;

68. The kit of paragraph 52, wherein the source of energy is a particle beam;

69. The kit of paragraph 52, wherein the source of energy is a source of electrical energy;

70. The kit of paragraph 52, wherein the source of energy is a source of alternating magnetic energy;

71. The kit of paragraph 52, wherein the source of energy is a source of photons;

72. A targetable particle comprising a magnetic component other than metallic iron and either carbon or ceramic material;

73. The targetable particle of paragraph 72, wherein the particle is a carbon-bearing particle;

74. The targetable particle of paragraph 73, wherein the carbon is chosen from the group consisting of activated carbon type A, type B, type E, type K, and type KB;

75. The targetable particle of paragraph 73, wherein the magnetic component is chosen from the group consisting of nickel, cobalt, awaruite, wairauite, pyrrhotite, greigite, troilite, yttrium iron garnet, Alnico 5, Alnico 5 DG, Sm₂Co₁₇, SmCo₅, and NdFeB components;

76. The targetable particle of paragraph 73, wherein the magnetic component is chosen from the group consisting of nickel, cobalt, awaruite, wairauite, pyrrhotite, greigite, troilite, and yttrium iron garnet components;

77. The targetable particle of paragraph 74, further comprising one or more biologically active agents;

78. The targetable particle of paragraph 77 wherein the one or more biologically active agents are chosen from the group consisting of antifungals, antibiotics and antineoplastic agents;

79. The targetable particle of paragraph 72, wherein the particle is a ceramic-bearing particle;

80. The targetable particle of paragraph 79, wherein the ceramic material is silica, octadecyl silica or other chemically modified silica, or hydroxyapatite;

81. The targetable particle of paragraph 79, wherein the magnetic component is chosen from the group consisting of nickel, cobalt, awaruite, wairauite, pyrrhotite, greigite, troilite, yttrium iron garnet, Alnico 5, Alnico 5 DG, Sm₂Co₁₇, SmCo₅, and NdFeB components;

82. The targetable particle of paragraph 79, wherein the magnetic component is chosen from the group consisting nickel, cobalt, awaruite, wairauite, pyrrhotite, greigite, troilite, and yttrium iron garnet components;

83. The targetable particle of paragraph 79, further comprising one or more biologically active agents;

84. The targetable particle of paragraph 83, wherein the one or more biologically active agents are chosen from the group consisting of antifungals, antibiotics and antineoplastic agents;

85. The targetable particle of paragraph 72, further comprising one or more biologically active agents;

86. The targetable particle of paragraph 85, wherein the one or more biologically active agents is chosen from the group consisting of antifungal, antibiotic and antineoplastic agents;

87. The targetable particle of paragraph 72, further comprising one or more excipients;

88. The targetable particle of paragraph 72, further comprising one or more delivery vehicles;

89. The targetable particle of paragraph 72 in a unit dose form.

Another embodiment includes a method of manufacture for any of the above targetable particles, which can further including milling said particles or treating them with gas as set forth below. Additionally, any of the above-listed targetable particles can further comprise being combined with one or more excipients and/or delivery agents. Any of these particles can be in a unit dosage form. Furthermore, any of the kits or methods of radiative therapy described herein can be used with any single magnetic component particle, optionally combined with any biologically active agent, excipient or delivery agent discussed herein.

In one embodiment, a method of radiative therapy is provided comprising the steps of introducing a magnetic composition into a patient, magnetically guiding the composition to a target, and depositing energy at the target site.

It is an advantage of the embodiment to provide for a desired type of placement of multiple particles that are able to apply a desired effect to local tissue, upon the application of deposited energy to the tissue containing the particles.

### BRIEF DESCRIPTION OF THE DRAWINGS

FIG. 1 depicts a magnetic resonance imaging (MRI) scan immediately following hepatic intra-arterial administration and magnetic localization of 50 mg of magnetocarbon particles. The circle indicates the target area showing uniform particle localization and retention.

FIG. 2 depicts a magnetic resonance imaging (MRI) scan of a human hepatocellular carcinoma after injection and magnetic localization of magnetocarbon particles. The circle indicates the target area showing uniform particle localization and retention. The light region in the center of the tumor is necrotic, as confirmed by computed tomography (CT) scan.

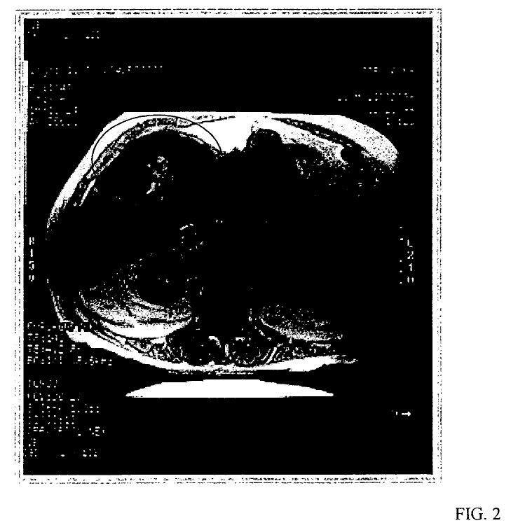

FIG. 3 is a magnified photograph (12000.times.) of magnetic component particles of this invention.

FIG. 4 is a magnified photograph (30,000.times.) of a magnetic component particle of this invention.

FIG. 5 is the magnetic saturation versus metallic iron content in particles.

FIG. 6 illustrates the magnetization curves of Bang's magnetite particles (NC05N) vs. metallic iron-based particles.

FIG. 7 illustrates the magnetic capture of magnetic particles in an in vitro experimental system.

### DETAILED DESCRIPTION OF THE PREFERRED EMBODIMENTS

In order to overcome the limitations of the current therapeutic options, this invention brings together a method for targeting magnetic component particles into a targeted site, and then depositing energy in a way to provide therapy to a localized disease.

The present embodiment relates to the method of use of magnetic component particle compositions for use in radiative procedures for medical use. Certain magnetic component compositions may be used successfully with the application of an externally placed non-alternating magnetic field that guides the particles to the desired biological target site, such as tissues, cells or cell components. The particles are extravasated into the target tissue, affording better localization and distribution of the particles throughout the tissue. Since the non-alternating magnetic guidance has no biological effect on healthy or diseased tissue, there is no barrier to targeting disease sites at any depth in the body. Then a radiative therapy is applied to the particles via the deposition of energy to the particles. The relatively small size and relatively even distribution of the particles yields a more uniform effect upon application of radiative therapy. The magnetic component particles may be used alone or in combination with one or more biologically active agents attached thereto, and in combination with other systemic and/or localized therapies.

The term “deposited energy” is meant to include the conversion or transfer of energy or mass. Deposited energy techniques include, but are not limited to gamma, beta, alpha, neutron, proton, X-ray, electron, and positron radiation, magnetic radiation, thermal radiation, microwave radiation, ultrasound radiation, ultraviolet, visible and infrared radiation, electric field radiation, and combinations thereof. The deposited energy may be in the form of a direct or alternating field, at any frequency, including but not limited to radio frequencies and microwave frequencies.

The term “targeted site” is meant to include any in vivo region of focal or localized disease. Targeted sites include, but are not limited to, tumors, malignancies, aneurysms, abscesses, infections, inflammations, viral growths, immunologically reactive sites, transplantation and implantation sites, joints, wounds, bones, specific organs or specific regions of the vasculature.

The term “magnetic component particle” is meant to include particles of specific composition, that composition being metallic iron, or another magnetic composition having a Curie temperature of >37° C. and a magnetic saturation greater than 20 A.m²/kg (emu/g). A magnetic component particle may optionally contain a portion of carbon (a “magnetocarbon” particle), ceramic (a “magnetoceramic” particle), or polymer (a “magnetopolymer” particle). Magnetocarbon and magnetoceramic particles together are “magnetosorbtive” particles. A magnetic component particle may also optionally contain a biologically active agent. A magnetic component particle may optionally be processed in one or more transformative manufacturing steps.

In all embodiments of the method of use, magnetic component particles are guided to the targeted site with a non-alternating magnetic field. Guidance may be further facilitated by a number of additional procedures, compositions or kits, which are described below.

Magnetic component particles may be guided to the targeted site while suspended in a delivery vehicle. One exemplary delivery vehicle is sterile saline. Other appropriate delivery vehicles have density and/or viscosity greater than that of water, to inhibit the settling and aggregation of magnetic component particles described herein. These include, but are not limited to aqueous polymer solutions, such as 0.1 to 3% carboxymethylcellulose of any molecular weight grade, 0.1 to 10% glycerol, and 0.1 to 20% polyethylene glycol, of any molecular weight grade or distribution. Polymers may be obtained from Shearwater Polymers (Huntsville Ala.), for example, or any other supplier of polymers having sufficient purity and consistency. Other vehicles may include sugar solutions that increase the viscosity and/or the density of the vehicle, such as mannitol, sucrose, glucose, lactose, and/or trehalose, in any concentration up to their solubility limits. These sugars can be obtained from virtually any supplier of specialty chemicals. Also useful are organic vehicles, such as oils, for example soybean oil, iodinated soybean oil (lipiodol), vegetable oil, peanut oil, etc. These oils typically have viscosity and/or density greater than that of water, and can be used parenterally under appropriate conditions. Organic vehicles of appropriate purity can also be obtained from multiple suppliers.

Because it is convenient to prepare and market the magnetic component particles in a dry form, the excipients may be prepared in dry form, and one or more dry excipients are packaged together with a unit dose of the magnetic component particles. A wide variety of excipients may be used, for example, to enhance precipitation or release of the biologically active agent, if present. A person having ordinary skill in the art readily can determine the types and amounts of appropriate dry excipients. The type and amount of appropriate dry excipients can readily be determined by any person having ordinary skill in the art. For instance, the excipients can be selected from a viscosity agent or a tonicifier, or both. Viscosity agents are, for example, biodegradable polymers such as carboxymethylcellulose, PVP, polyethylene glycol (PEG), and the like. Tonicifiers include sodium chloride, mannitol, dextrose, lactose, and other agents used to impart the same osmolarity to the reconstituted solution. Most preferably, the package or kit containing both the dry excipients and dry magnetic particles such as iron is formulated to be mixed with the liquid contents of a vial containing a unit dose of the biologically active compound. Liquid agents could be used as excipients just prior to use of the particles. Such liquid agents could be soybean oil, rapeseed oil, or an aqueous based polymer solution composed of the polymers as listed above. Also liquid solutions could be a tonicifier, such as Ringer's solution, 5% dextrose solution, physiological saline. As before a combination of liquid excipients and tonicifiers can be used. (See, for example, Kibbe, A H, Handbook of Pharmaceutical Excipients, American Pharmaceutical Association, Washington, D.C., 2000), herein incorporated by reference). Upon mixture of the liquid containing the biologically active compound with the contents of the kit including the dry components (i.e., the dry iron particles and dry excipients), the biologically active compound attaches to the magnetic particles according to a protocol developed for each compound, thus forming a magnetically controllable composition containing a diagnostic and/or therapeutic amount of a biologically active compound attached to the magnetic particles and being suitable for ex vivo or in vivo therapeutic and/or diagnostic as well as ex vivo diagnostic use. Any suitable sterilization technique may be employed. For example, iron particles may be sterilized using gamma or electron irradiation or dry heat and the aqueous solution of excipients may be sterilized by autoclave. The resulting particles having attached thereon one or more biologically active compounds (“magnetically susceptible compositions”) may be used alone or incorporated into a delivery system. Suitable delivery systems will be apparent to any person possessing ordinary skill in the art. Without limitation, examples of useful delivery systems include matrices, capsules, slabs, microspheres, and liposomes. Conventional excipients may be incorporated into any of the formulations. Most preferably, the package or kit containing both the dry excipients and dry magnetic component particles is formulated to be mixed with the liquid contents of a vial containing a unit dose of the biologically active agent, if desired. Such kits, containing a biologically active agent, are discussed more fully below.

The assays or therapies may involve a kit. In one embodiment, the package or kit contains both dry excipients and dry magnetic component particles, formulated to be mixed with the liquid contents of a vial containing a unit dose of a biologically active agent. Upon mixture of the liquid containing the biologically active agent with the contents of the kit including the dry components (i.e., the dry magnetic component particles and dry excipients), the biologically active agent attaches to the magnetic component particles according to a protocol developed for each agent. Any suitable sterilization technique may be employed. For example, the magnetic component may be sterilized using gamma radiation, and the aqueous solution of excipients may be sterilized by autoclave.

The methods of use include methods for localized in vivo treatment of disease using a magnetic component particle, optionally having precipitated thereon one or more biologically active agents selected for efficacy in treating the disease, magnetically guiding the particle to a desired location in the body of a patient, and depositing energy to the desired location. The particles may be introduced by injection, infusion, implantation, ingestion, or other routes of administration whereby the particles are delivered to the inside of the body.

Introduction of the particles into the body of a patient can be achieved in a variety of routes, including, but without limitation to, intra-arterial, intra-venous, intra-tumoral, intra-peritoneal, and subcutaneous. For example, the magnetic component particles described herein may be injected by inserting delivery means, such as a catheter or needle, into an artery within a short distance from a body site to be treated and at a branch or branches, preferably the most immediate, to a network of arteries carrying blood to the site. The particles are injected through the delivery means into the blood vessel.

Just prior to, during or after injection, a non-alternating magnetic field is established exterior to the body and adjacent to the targeted site, and having sufficient field strength to guide a substantial quantity of the injected magnetic component particles to, and retain the substantial quantity of the particles at the site. Preferably, the magnetic field is of sufficient strength to draw the magnetic component particles into the soft tissue at the site adjacent to the network of vessels, thus avoiding substantial embolization of any of the larger vessels by the particles, should embolization be undesirable for the particular treatment/diagnosis. Examples of such magnets for use in the instant methods are those producing at least about 100 gauss of non-alternating magnetic flux at the region of interest (target site), the exact magnetic field strength being dependent upon the application, for instance the blood flow rate, the thickness of the endothelium, and the depth and diffuseness of the tumor tissue. For example, an NdFeB magnet producing a flux of about 5 kG at its N pole surface, having a dimension of about 5 cm diameter, 6 cm length, can be used to direct particles described herein in both healthy and diseased liver tissue. (Part No. MSD12691-NC, Magnet Sales, Culver City, Calif.). Other compositions of NdFeB, and other rare earth, ceramic, or electromagnets or superconducting magnets may also be suitable.

There are many alternative mechanisms for guiding the magnetic component particles to the desired region in the host. Which approach is desirable for a given situation will depend upon the goal to be achieved, given the present disclosure, one of skill in the art will be able to readily determine which approach should be used. In one embodiment, the magnetic component particles are directed and controlled by the invention of Mitchiner et al., U.S. Pat. No. 6,488,615, issued Dec. 3, 2002, herein incorporated in its entirety by reference. This reference provides both the device for administering a magnetic field to a patient in order to capture these particles, and the method for doing so. Briefly, the device is a magnet keeper-shield assembly adapted to hold and store a permanent magnet used to generate a high gradient magnetic field. Such a field may penetrate into deep targeted tumor sites in order to attract magnetic component particles. The magnet keeper-shield assembly includes a magnetically permeable keeper-shield with a bore dimensioned to hold the magnet. An actuator is used to push the magnet partially out of the keeper-shield. The actuator is assisted by several springs extending through the base of the keeper-shield.

In another embodiment, the magnetic component particles are injected into a targeted site and the magnetic field is used to redistribute the particles in an appropriate manner to achieve the desired effect. In some situations, the important feature of the embodiment will be the ability to distribute the particles throughout a tissue, in a relatively even manner, thus allowing for even treatment of the tissue and for full destruction of the tissue, with minimal damage to all of the surrounding tissue. In an alternative embodiment, the particles are guided in a manner to reduce damage to a particular neighboring tissue. For example, while it may be desirable to eliminate the tissue in question, there may be a part of the tissue that is in close proximity to a tissue or organ that is crucial to survival and thus is especially sensitive to damage. In these circumstances, it may be desirable to sacrifice an even distribution of the particles in order to prevent excessive damage to crucial tissues, while still getting some of the benefits of this embodiment.

In some cases, embolism may be desired, as the deposited energy necessary to result in tissue damage is often decreased when used in conjunction with an embolic agent. If such an embolic agent is desired, the magnetic component particles of the invention may be used. For example, particles prepared in the size range from about 20 μm to about 50 μm may be prepared by the methods described above and also guided to the targeted site and held in place by an external non-alternating magnet. In order to provide optimal embolization and extravasation, two batches of the particles may be prepared in different sizes, the larger size being used as the embolic agent. Alternatively, other embolic agents may be used in conjunction with the methods of this invention and are well known in the art. Examples of embolic devices include, but are not limited to, balloon catheters, and gelatin sponge particles (Gelfoam®, Pfizer, Kalamazoo, Mich.).

In the case where the biologically active agent(s) includes a diagnostic imaging agent, the imaging is performed while the magnetic component particles are captured at the targeted site, and in some cases before, during and/or after. For example, a mapping procedure may be performed prior to the deposited energy procedure, or imaging may be desirable throughout the procedure. Imaging is often desired subsequent to the procedure to immediately assess the amount of success.

Imaging modalities and methods are well known to any person having ordinary skill in the art and include, but are not limited to ultrasound, x-ray, magnetic resonance imaging, positron emission tomography and computed tomography.

One embodiment generally involves injection of the magnetic component particles, optionally having an attached biologically active agent, and optionally in conjunction with an imaging modality; guiding and maintaining the magnetic component particles at the targeted site using an externally placed non-alternating magnetic field, optionally in conjunction with an embolic agent; and applying deposited energy, optionally in conjunction with one or more therapies designed to induce a therapeutic effect, for example tissue damage or coagulation at the targeted site in the case of neoplasm. The deposited energy should be applied so as to maintain a therapeutic effect for a sustained period, for example in the case of hyperthermia, temperature of from about 40° C. to about 50° C. for a period of from about 30 minutes to about 3 hours. Doses of deposited energy and/or ultimate temperature may be lower where use of an embolic agent is also employed, due in part to the lack of cooling provided by circulating blood. Any number of procedures may be combined, such as the use of ionizing radiation and/or chemotherapy. Optional radiation protective devices or compositions, such as those for cooling, may be employed in order to protect the area surrounding the targeted site.

In one embodiment, the “magnetic component” of the magnetic component particles comprises a metallic iron that is relatively free of iron oxides, as described below. In one embodiment, the magnetic component has the general properties of having Curie temperatures (Tc) greater than the normal human body temperature (37° C.), having high magnetic saturation (>approximately 20 Am²/kg), and being ferromagnetic or ferrimagnetic. Examples of suitable magnetic components include magnetic iron sulfides such as pyrrhotite (Fe₇S₈), and greigite (Fe₄S₄), magnetic ceramics such as Alnico 5, Alnico 5 DG, Sm₂Co₁₇, SmCo₅ and NdFeB, magnetic iron alloys, such as jacobsite (MnFe₂O₄), trevorite (NiFe₂O₄), awaruite (Ni₃Fe) and wairauite (CoFe), and magnetic metals such as metallic iron (Fe, ⁵⁹Fe), cobalt (Co, ⁵⁵Co, ⁵⁶Co), nickel (Ni, ⁵⁷Ni). Each of the magnetic components can have added to its chemical formula specific impurities that may or may not alter the magnetic properties of the material. Doped ferromagnetic or ferrimagentic materials within the above limits of Curie temperatures and magnetic saturation values are considered to be within the scope of the instant embodiment. Specifically excluded from the magnetic components and the magnetic component particles of the instant invention are the “iron oxides” magnetite (Fe₃O₄), hematite (αFe₂O₃), and maghemite (γFe₂O₃).

One exemplary group of magnetic components for use in the magnetic component particles is selected from the group consisting of iron, nickel, awaruite, wairauite, pyrrhotite, greigite, troilite, yttrium iron gamet, Alnico 5, Alnico 5 DG, Sm₂Co₁₇, SmCo₅, and NdFeB particles. Another exemplary group for use in the magnetic component particles to be used in this embodiment is selected from the group consisting of iron, nickel, awaruite, wairauite, pyrrhotite, greigite, troilite, and yttrium iron gamet particles. Another group for use in the magnetic component particles to be used in this embodiment are those that have a magnetic saturation value of greater than or equal to 20 A.m²/kg, excluding metallic iron and iron oxides. Yet another exemplary group for use in the magnetic component particles to be used in this embodiment are ferrimagnetic.

One embodiment of magnetic component particles is targetable particles. Targetable particles are those magnetic component particles that comprise one or more magnetic components of the magnetic component particles except for metallic iron and also comprise either the carbon or the ceramic materials as set forth for the magnetic component particles. These targetable particles are another aspect of the instant invention. Examples of the carbon-bearing targetable particles are those containing carbon and a magnetic component chosen from the group consisting of nickel, cobalt, awaruite, wairauite, pyrrhotite, greigite, troilite, yttrium iron garnet, Alnico 5, Alnico 5 DG, Sm₂Co₁₇, SmCo₅, and NdFeB components. Another exemplary group of the carbon-bearing targetable particles include those comprising nickel, cobalt, awaruite, wairauite, pyrrhotite, greigite, troilite, and yttrium iron garnet as well as carbon. Similarly, examples of the ceramic-bearing targetable particles include those comprising a ceramic material and a magnetic component chosen from the group consisting of nickel, cobalt awaruite, wairauite, pyrrhotite, greigite, troilite, yttrium iron garnet, Alnico 5, Alnico 5 DG, Sm₂Co₁₇, SmCo₅, and NdFeB particles. Yet another such group of ceramic-bearing targetable particles are those comprising a magnetic material chosen from the group consisting of nickel, cobalt, awaruite, wairauite, pyrrhotite, greigite, troilite, and yttrium iron garnet and a ceramic material. Examples of the ceramic materials include silica, octadecylsilica or other chemically modified silica and hydroxyapatite. Examples of the carbon include one or more chosen from the group consisting of activated carbon type A, type B, type E, type K, and type KB. The targetable particles can be made by mechanical milling, including planetary milling, attrition milling and other forms of high energy milling, and are subject to the same size, Curie temperature and magnetic saturation constraints that apply for the magnetic component particles. The targetable particles can be combined with one or more biologically active agents as described below.

Further examples of such biologically active agents are chosen from the group consisting of antineoplastics, antibiotics, and antifungals. One example of such a biologically active agent is doxorubicin. The targetable particles can be combined with one or more excipients, as described herein, and be a part of a kit, as described herein.

In one embodiment, “metallic iron” used for making the particles that are used in this embodiment is essentially chemically pure, with higher than about 85% atomic iron, and most preferably higher than about 90%. The iron used for making the particles used in this embodiment also typically contains less than about 20% iron oxides, more preferably less than about 10%, and most preferably less than about 5%, it being noted that the particles may contain impurities in addition to iron oxides. Metallic iron is a material with high magnetic saturation and density (218 emu/g and 7.8 g/cm³) which are much higher than magnetite (92 emu/g and 5.0 g/cm³). The density of metallic iron is 7.8 g/cm³, while magnetite is about 5.0 g/cm³. Thus, the magnetic saturation of metallic iron is about 4-fold higher than that of magnetite per unit volume. (CRC Handbook, 77th edition, CRC Press (1996-1997) and Craik, D., Magnetism Principles and Applications, Wiley and Sons (1995).

Because the iron in the magnetic component particles described is not in the form of an iron oxide, as is the case in certain previously disclosed magnetically controlled dispersions, the magnetic susceptibility, or responsiveness, of the particles is maintained at a high level.

All magnetic component particles described herein possess superior magnetic susceptibility. The “magnetic susceptibility” of the particles is the degree of magnetic responsiveness of the particles to a magnetic field, wherein lack of magnetic susceptibility correlates to an absence of response to a magnetic field. This responsiveness may be affected for example, by the components present in the magnetic component particle composition, by the route of administration, by the resulting depth of the particles in the body and/or strength of the magnetic field.

The magnetic component for the particles may be purchased in powder form. Suitable magnetic component powder is preferably in the nanometer to micron size range (for example, Iron, ISP, Wayne, N.J.).

The upper limit to the magnetic component particle size is the diameter of the vessels of vasculature into which the particles are injected. This diameter varies within the human anatomy, from 5 cm to on the order of 5 μm. Thus, there are applications for particles of virtually all sizes below 5 cm. The majority of non-embolic applications will be for particles of 0.1 to 20 μm, with the most favored size range from 0.5 μm to 5 um. In the case where blood vessel embolization is desired, particles from about 2 μm to 5 cm are desirable, while most applications can be satisfied with 5 μm to 100 μm particles with most preferred from 10 μm to 50 μm. Of course, as will be appreciated by one of skill in the art, the sizes of the particles need not be identical within the population of particles administered.

In one embodiment, the magnetic component compositions to be used herein may include nonmagnetic materials and thus be magnetocarbon and magnetoceramic particles. These particles may also have one or more optionally attached biologically active agents.

In one embodiment, the magnetic component particles are comprised of a magnetoceramic material, and are comprised of up to 95.0% ceramic (or a ceramic derivative) and the balance magnetic component, by mass. With compositions of greater than 95.0% ceramic, the magnetic susceptibility is generally reduced beyond an effective level for targeting biologically active agents in vivo. It is important to realize that the particles are to be directed by a magnetic field, as such, they generally should be of sizes previously disclosed. For a full description of such particles, see Rudge et al., WO/01/28587, published 26 Apr., 2001, herein incorporated in its entirety by reference.

The term “ceramic” for the magnetoceramic particles means a natural or synthetic porous, adsorptive material. It is usually, but not necessarily an oxide or mixed oxide, wherein the oxide is metallic or non-metallic. It is usually, but not necessarily inorganic. It is usually, but not necessarily without a crystalline structure. Examples of ceramic materials include, but are not limited to tricalcium phosphate, hydroxyapatite, aluminum hydroxide, aluminum oxide, aluminum calcium phosphate, dicalcium phosphate dihydrate, tetracalcium phosphate, macroporous triphasic calcium phosphate, calcium carbonates, hematite, bone meal, apatite wollastonite glass ceramics and other ceramic or glass matrices. Appropriate materials based upon these parameters will be apparent to any person having ordinary skill in the art. A table of examples follows.

| | Oxide | Non-metallic | Amorphous |
|---|---|---|---|
| Silica | Y | Y | Y |
| Hydroxyapatite | Y | N | Y |
| Zeolites | Y | N | N |
| Aluminas | Y | N | Y |
| Diamond | N | Y | N |

Also included in the definition of “ceramic” for the magnetoceramic particles are silica and silica derivatives (including, but not limited to octadecycl silane [C₁₈], octyl silane [C₈], hexyl silane [C₆], phenyl silane [C₆], butyl silane [C₄], aminopropylsilane [NH₃C₃], cyano nitrile silane [CN], trimethylsilane [C,], sulfoxyl propyl silane [SO₄C₃], dimethylsilane [C₁], acidic cation-exchange coating [SCX], basic quaternary ammonium anion exchange coating [SAX], dihydroxypropyl silane [diol]), into a particle. By way of example, the following silicas are useful for forming the particles to be used in the embodiments of the present invention.

EKA NOBEL KROMASIL ®

| | | | | | | | Bonded | |
|---|---|---|---|---|---|---|---|---|
| | Particle | | Pore | Surface | | | Phase | |
| Packing | Shape & | Pore Size | Volume | Area | Carbon | | Coverage | End |
| Material | Size (μm) | (Å) | (ml/g) | (m²/g) | Load (%) | Phase Type | (μmol/m²) | Cap |
| Kromasil | S, 5, 7, 10, | 100 | 0.9 | 340 | — | (elemental | | |
| Silica | 13, 16 | | | | | analysis) | | |
| Kromasil | S, 5, 7, 10, | 100 | 0.9 | 340 | 4.7 | Monomeric | 4.3 | — |
| C1 | 13, 16 | | | | | | | |
| Kromasil | S, 5, 7, 10, | 100 | 0.9 | 340 | 8 | Monomeric | 3.7 | Yes |
| C4 | 13, 16 | | | | | | | |
| Kromasil | S, 5, 7, 10, | 100 | 0.9 | 340 | 12 | Monomeric | 3.6 | Yes |
| C8 | 13, 16 | | | | | | | |
| Kromasil | S, 5, 7, 10, | 100 | 0.9 | 340 | 19 | Monomeric | 3.2 | Yes |
| C18 | 13, 16 | | | | | | | |

EMD CHEMICALS

| | | | | | | | Bonded | |
|---|---|---|---|---|---|---|---|---|
| | Particle | | Pore | Surface | | | Phase | |
| Packing | Shape & | Pore Size | Volume | Area | Carbon | | Coverage | End |
| Material | Size (μm) | (Å) | (ml/g) | (m²/g) | Load (%) | Phase Type | (μmol/m²) | Cap |
| Lichrosorb | I, 5, 10 | 60 | — | 550 | 0 | — | — | No |
| Si 60 | | | | | | | | |
| Lichrosorb | I, 5, 10 | 100 | — | 420 | 0 | — | — | No |
| Si 100 | | | | | | | | |
| Lichrosorb | I, 5, 10 | 60 | — | 150 | 16.0 | Monomeric | 1.55 | No |
| RP-18 | | | | | | | | |
| Lichrosorb | I, 5, 10 | 60 | — | — | 9.0 | Monomeric | 0.78 | No |
| RP-8 | | | | | | | | |
| Lichrosorb | I, 5, 10 | 60 | 0.7 | 550 | 12 | — | 2.5 | Yes |
| RP-select B | | | | | | | | |
| Lichrospher | S, 3, 5, | 60 | 0.95 | 650 | 0 | — | 0 | No |
| Si 60 | 10 | | | | | | | |
| Lichrospher | S, 5, 10 | 100 | 1.25 | 420 | 0 | — | 0 | No |
| Si 100 | | | | | | | | |
| Lichrospher | S, 3, 5, | 60/100 | 1.25 | 350 | 12.5 | — | 4.1 | No |
| RP-8 | 10 | | | | | | | |
| Lichrospher | S, 3, 5, | 60/100 | 1.25 | 350 | 13 | — | 4.2 | Yes |
| RP-8 E/C | 10 | | | | | | | |
| Lichrospher | S, 3, 5, | 100 | 1.25 | 350 | 21.4 | — | 3.9 | No |
| RP-18 | 10 | | | | | | | |
| Lichrospher | S, 3, 5, | 100 | 1.25 | 350 | 21.5 | — | — | Yes |
| RP-18 E/C | 10 | | | | | | | |
| Lichrospher | S, 3, 5, | 100 | 1.25 | 350 | — | — | — | — |
| CN | 10 | | | | | | | |
| Lichrospher | S, 3, 5, | 100 | 1.25 | 350 | 4.5 | — | 3.8 | — |
| NH2 | 10 | | | | | | | |
| Lichrospher | S, 3, 5, | 100 | 1.25 | 350 | 8.3 | — | 4.0 | — |
| Diol | 10 | | | | | | | |
| Lichrospher | S, 3, 5, | 60 | 0.9 | 360 | 12.0 | — | 3.2 | Yes |
| RP-select B | 10 | | | | | | | |
| Inertsil | S, 5 | 150 | — | 320 | 0 | — | — | No |
| Silica | | | | | | | | |
| Inertsil | S, 5 | 150 | — | 320 | 18.5 | Monomeric | 3.23 | Yes |
| ODS-2 | | | | | | | | |
| Inertsil | S, 3, 5 | 100 | — | 450 | 15 | Monomeric | — | — |
| ODS-3 | | | | | | | | |
| Inertsil C8 | S, 5 | 150 | — | 320 | 10.5 | Monomeric | 3.27 | Yes |
| Inertsil C8-3 | S, 5 | 100 | — | 450 | 10 | Monomeric | — | Yes |
| Inertsil Ph | S, 5 | 150 | — | 320 | 10 | Monomeric | 2.77 | Yes |
| (Phenyl) | | | | | | | | |
| Inertsil Ph-3 | S, 5 | 100 | — | 450 | 10 | Monomeric | — | Yes |
| (Phenyl) | | | | | | | | |
| Inertsil C4 | S, 5 | 150 | — | 320 | 7.5 | Monomeric | 3.77 | Yes |
| Inertsil 80 Å | S, 5 | 80 | — | 450 | 16 | Monomeric | — | Yes |
| Inertsil Prep | S, 10 | 100 | — | 350 | 14 | — | — | — |
| ODS, C8, Si | | | | | | | | |

WATERS ASSOCIATES

| | | | | | | | Bonded | |
|---|---|---|---|---|---|---|---|---|
| | Particle | | Pore | Surface | | | Phase | |
| Packing | Shape & | Pore Size | Volume | Area | Carbon | | Coverage | End |
| Material | Size (μm) | (Å) | (ml/g) | (m²/g) | Load (%) | Phase Type | (μmol/m²) | Cap |
| μBondapak | I, 10 | 125 | 1.0 | 330 | 10 | Monomeric | 1.46 | Yes |
| C18 | | | | | | | | |
| μBondapak | I, 10 | 125 | 1.0 | 330 | 8 | — | 2.08 | Yes |
| Phenyl | | | | | | | | |
| μBondapak | I, 10 | 125 | 1.0 | 330 | 3.5 | — | 1.91 | No |
| NH2 | | | | | | | | |
| μBondapak | I, 10 | 125 | 1.0 | 330 | 6 | — | 2.86 | Yes |
| CN | | | | | | | | |
| μPorasil | I, 10 | 125 | 1.0 | 330 | — | — | — | No |
| Silica | | | | | | | | |
| Novapak | S, 4 | 60 | 0.3 | 120 | 7 | — | 3.41 | Yes |
| C18 | | | | | | | | |
| Novapak | S, 4 | 60 | 0.3 | 120 | 5 | — | 2.34 | Yes |
| Phenyl | | | | | | | | |
| Novapak | S, 4 | 60 | 0.3 | 120 | 2 | — | 1.65 | Yes |
| CN | | | | | | | | |
| Novapak | S, 4 | 60 | 0.3 | 120 | 0 | — | 0 | No |
| Silica | | | | | | | | |
| Resolve | S, 5, 10 | 90 | 0.5 | 175 | 10 | — | 2.76 | No |
| C18 | | | | | | | | |
| Resolve C8 | S, 5, 10 | 90 | 0.5 | 175 | 5 | — | 2.58 | No |
| Resolve CN | S, 5, 10 | 90 | 0.5 | 175 | 3 | — | 2.53 | No |
| Resolve | S, 5, 10 | 90 | 0.5 | 175 | 0 | — | 0 | No |
| Silica | | | | | | | | |
| Spherisorb | S, 3, 5, | 80 | 0.5 | 220 | 0 | — | 0 | No |
| Silica | 10 | | | | | | | |
| Spherisorb | S, 3, 5, | 80 | 0.5 | 220 | 7 | Monomeric | 1.47 | Partial |
| ODS-1 | 10 | | | | | | | |
| Spherisorb | S, 3, 5, | 80 | 0.5 | 220 | 12 | Monomeric | 2.72 | Yes |
| ODS-2 | 10 | | | | | | | |
| Spherisorb | S, 3, 5, | 80 | 0.5 | 220 | 6 | Monomeric | 2.51 | Yes |
| C8 | 10 | | | | | | | |
| Spherisorb | S, 3, 5, | 80 | 0.5 | 220 | 6 | Monomeric | 2.27 | Yes |
| C6 | 10 | | | | | | | |
| Spherisorb | S, 3, 5, | 80 | 0.5 | 220 | 3 | Monomeric | 1.08 | Partial |
| Phenyl | 10 | | | | | | | |
| Spherisorb | S, 3, 5, | 80 | 0.5 | 220 | 3.5 | Monomeric | 2.37 | No |
| CN | 10 | | | | | | | |
| Spherisorb | S, 3, 5, | 80 | 0.5 | 220 | 2 | Monomeric | 1.58 | No |
| NH2 | 10 | | | | | | | |
| Spherisorb | S, 5, 10 | 80 | 0.5 | 220 | — | — | — | No |
| SAX | | | | | | | | |
| Spherisorb | S, 5, 10 | 80 | 0.5 | 220 | — | — | — | — |
| SCX | | | | | | | | |
| Symmetry | S | 100 | — | 340 | 19 | — | 3.09 | Yes |

Note:
Bonded phase coverage calculated as per Sander, L. C., and Wise, S. A., Anal. Chem, 56: 504-510, 1984.
Material characteristics obtained from literature published by the material manufacturer or an authorized representative thereof.
EKA Nobel Kromasil (Goteborg, Sweden), EM Science (Darmstadt, Germany), Waters Associate (Bedford, Mass.).

In an alternative embodiment, magnetocarbon magnetic component particles may be made and used according to this invention. Raw carbon granules may be used for making the particles. Most preferred are activated carbon types A, B, E, K and KB (Norit Americas, Inc., Norcross, Ga.). For a detailed discussion of magnetocarbon component particles, see Volkonsky et al., U.S. Pat. No. 6,482,436, issued Nov. 19, 2002, herein incorporated by reference in its entirety.

In one embodiment, the magnetic component particles include volume-compounded magnetocarbon particles, containing about up to about 95.0% by mass of carbon, for example, between about 10% and 60%. About 20% to about 40% is the preferred range of carbon having been found to exhibit characteristics useful in many applications.

The magnetic component particles may comprise raw magnetic component particles or processed magnetic component particles. Use of either raw or processed magnetic component particles can affect the adsorption, precipitation, or labeling of biologically active agents onto the microparticles, as well as affecting the stability as a function of time, and the magnetic susceptibility. Depending on the desired characteristics, processes might be used singly or in combination. Processes that may be employed include milling, chemical vapor deposition, or gas phase treatment. (See, e.g., Reynoldson, R. W. Heat Treatment of Metals, 28:15-20 (2001); Ucisik et al., J. Australasian Ceramic Soc., 37, (2001); Isaki et al., Japanese Patent 08320100 (1996); and Pantelis et al., “Large scale pulsed laser surface treatment of a lamellar graphite cast iron”, Surface Modification Technologies VIII. Proceedings, 8^{th} International Conference, Nice, France, 26-28 Sep. 1994, eds. T. S. Sudarshan, M. Jeandin, J. J. Stiglich, W. Reitz. Publ: London SW1Y 5 DB, UK The Institute of Materials, 297-309 (1995)). Other suitable processes are apparent to those having skill within the art.

Magnetic component particles may be processed in manners not likely to result in formation of iron oxides, such as would occur with application of extreme heat or certain chemical processes that are easily discernable to a person having ordinary skill within the art. Preferred processes include high-energy milling or gas or liquid phase treatment. It is believed that subjecting the magnetic component to high-energy milling may increase the magnetic susceptibility of the particles and/or lead to other desirable properties.

The magnetic component particle surface may be optimized, for example, to enhance binding of biologically active agents, as further discussed below.

If desired, the magnetic component particles can be processed to change their shape, size, surface area, and surface chemistry before being incorporated into a vehicle, or where desired, before biologically active agents are labeled, adsorbed or precipitated thereon, such processes being generally well known in the art. Many different processes can be used to increase and to optimize either the magnetic susceptibility of the magnetic component particles or the resulting amount of the biologically active agents that can be associated with the magnetic component particles. For example, raw magnetic component microparticles can undergo gas phase treatment or activation, milling, thermal activation, chemical vapor deposition of functional groups or any of a variety of other techniques apparent to any person skilled in the art.

For example, but without limitation, the magnetic component particles may be milled, as described below. This milling step may result in particles with higher magnetic susceptibility because of the particles' deformations during the process.

The high-energy milling process consists of combining the magnetic powder and optionally carbon and/or ceramic with a liquid, for example ethanol, in a canister containing grinding balls. The liquid serves as a lubricant during the milling process and also inhibits the oxidation of the powder; an especially important consideration when fabricating magnetic particles comprising the magnetic component. The canisters are then placed in a laboratory planetary mill of the type characteristically used in metallurgy (e.g. Pulversette, Fritsch, Albisheim, Germany). Other types of mills producing similar results may also be employed. The mill is run for an appropriate time (generally between 1 and 10 hours) at speeds, for example, between 100 rpm and 1000 rpm. At the end of the cycle, the magnetic component particles are collected. The magnetic component particles may be re-suspended and homogenized if desired. The magnetic component particles may be dried by any suitable technique, allowing for the protection of the material against oxidation.

Another process includes subjecting the magnetic component particles to a gas phase treatment. For example, the magnetic component particles may be placed in a quartz container within an oven. Hydrogen may be used to replace air in the oven and the temperature is then raised for example, to about 300° C. The magnetic component particles are left in this environment for about 2 hours. At the end of the cycle, the temperature is lowered and hydrogen is replaced by nitrogen. Once the magnetic component particles' temperatures have been returned to room temperature, they are collected and packaged. This process results in an increase in the roughness of the magnetic component particle's surface, leading to enhanced attachment of a biocompatible polymer and a biologically active agent, embodiments that will be more fully discussed below

The magnetic component particles may be optionally washed, dried, recovered, sterilized and/or filtered. Routine methods of packaging and storing may be employed. For example, the raw or processed dried magnetic component or magnetic component particles may be packaged in appropriate container closure system, for example, one enabling unit dosage forms. Packaging under nitrogen, argon or other inert gas is preferred to limit the oxidation of the magnetic component. Although the particles may be stored “wet,” it is preferred that the liquid should not be aqueous. For example, ethanol or DMSO may be employed. The particles may be sterilized by any appropriate means, keeping in mind that some methods may tend to undesirably lead to oxidation of the particles.

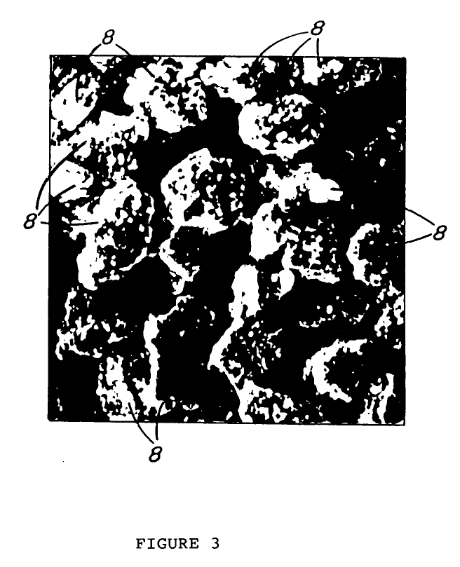

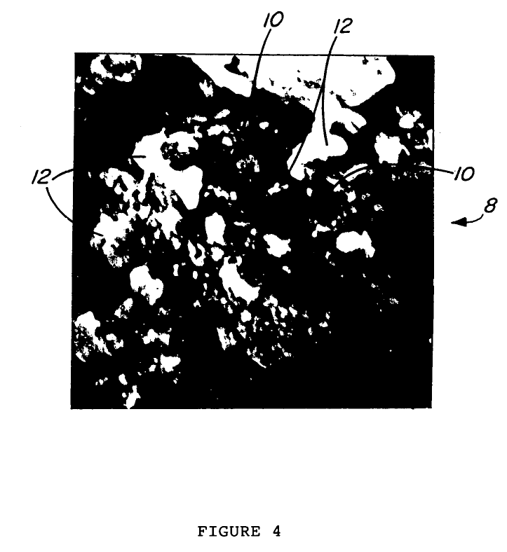

As shown in FIGS. 3 and 4, magnetocarbon particles 8 manufactured by the method of this invention are of a generally spherical shape, with the inclusions of carbon deposits 10 presumably being located randomly throughout the volume of each particle. The strong connection between the components (magnetic component 12 and carbon 10) is not broken during prolonged storage of the magnetically controlled composition, its transportation, storing, packing and direct use. Chemical binding may take place between the magnetic component and carbon, such as a trace interlayer of cementite (Fe₃C) formed during the milling process.

The magnetocarbon magnetic component particles are also useful as a carrier for delivering one or more adsorbed biologically active agents to targeted sites of the patient under control of an external magnetic field. As used herein, the term “biologically active agent” is as described below.

As a general principle, the amount of any aqueous soluble biologically active agent adsorbed can be increased by increasing the proportion of carbon in the magnetocarbon particles up to a maximum of about 40% by mass of the particles without loss of utility of the particles in the therapeutic treatment regimens described in this application. In many cases it has been observed that an increase in the amount of adsorbed biologically active agent is approximately linear with the increase in carbon content. However, as carbon content increases, the susceptibility, or responsiveness, of particles to a magnetic field decreases, and thus conditions for their guidance in the body worsen (although adsorption capacity increases). Therefore, it is necessary to achieve a balance in the magnetic component:carbon ratio to obtain improved therapeutic or diagnostic results. To increase the amount of agent given during a treatment regimen, a larger dose of particles can be introduced to the patient, but the particles cannot be made more magnetic by increasing the dose. Appropriate ratios may be determined by any person having average skill in the art.

It has been determined that the useful range of magnetic component:carbon ratio for the magnetocarbon particles intended for use in in vivo therapeutic treatments as described in the application is, as a general rule, from about 95:5 to about 50:50, for example about 80:20 to about 60:40. The maximum amount of the biologically active agent that can be adsorbed in the magnetocarbon component particles of any given concentration of carbon will also differ depending upon the chemical nature of the biologically active agent, and, in some cases, the type of carbon (i.e., activated carbon (AC)) used in the composition. For example, it has been discovered that the optimal magnetic component:carbon ratio for magnetocarbon particles used to deliver adsorbed doxorubicin in in vivo therapeutic treatments is about 75:25.

The magnetocarbon and magnetoceramic (“magnetosorptive”) magnetic component particles may be made in any manner that does not result in substantial production of iron oxides. The term “magnetosorptive” is defined as any combination of magnetic component and an adsorptive phase in a composite. A method for producing the particles is the high-energy milling method described above, whereby both the magnetic component and the adsorptive phase are used as starting materials at the onset of the process. As is well known in the art, the milling method provides volume compounded magnetosorptive particles, which may be used alone or in combination with one or more attached biologically active agents.

Another magnetic component particle for use in the present embodiment further comprises a biocompatible polymer, also referred to herein as “magnetopolymer particles,” as will be more filly disclosed below.

The term “biocompatible polymer” for the magnetopolymer particles is meant to include any synthetic and/or natural polymer that can be used in vivo. The biocompatible polymer may be bioinert and/or biodegradable. Some non-limiting examples of biocompatible polymers are polylactides, polyglycolides, polycaprolactones, polydioxanones, polycarbonates, polyhydroxybutyrates, polyalkylene oxalates, polyanhydrides, polyamides, polyacrylic acid, poloxamers, polyesteramides, polyurethanes, polyacetals, polyorthocarbonates, polyphosphazenes, polyhydroxyvalerates, polyalkylene succinates, poly(malic acid), poly(amino acids), alginate, agarose, chitin, chitosan, gelatin, collagen, atelocollagen, dextran, proteins, and polyorthoesters, and copolymers, terpolymers and combinations and mixtures thereof.

The biocompatible polymers for the magnetopolymer particles can be prepared in the form of matrices. Matrices are polymeric networks. One type of polymeric matrix is a hydrogel, which can be defined as a water-containing polymeric network. The polymers used to prepare hydrogels can be based on a variety of monomer types, such as those based on methacrylic and acrylic ester monomers, acrylamide (methacrylamide) monomers, and N-vinyl-2-pyrrolidone. Hydrogels can also be based on polymers such as starch, ethylene glycol, hyaluran, chitose, and/or cellulose. To form a hydrogel, monomers are typically crosslinked with crosslinking agents such as ethylene dimethacrylate, N,N-methylenediacrylamide, methylenebis(4-phenyl isocyanate), epichlarohydin glutaraldehyde, ethylene dimethacrylate, divinylbenzene, and allyl methacrylate. Hydrogels can also be based on polymers such as starch, ethylene glycol, hyaluran, chitose, and/or cellulose. In addition, hydrogels can be formed from a mixture of monomers and polymers.

Another type of polymeric network for the magnetopolymer particles can be formed from more hydrophobic monomers and/or macromers. Matrices formed from these materials generally exclude water. Polymers used to prepare hydrophobic matrices can be based on a variety of monomer types such as alkyl acrylates and methacrylates, and polyester-forming monomers such as ε-caprolactone, glycolide, lactic acid, glycolic acid, and lactide. When formulated for use in an aqueous environment, these materials do not need to be crosslinked, but they can be crosslinked with standard agents such as divinyl benzene. Hydrophobic matrices can also be formed from reactions of macromers bearing the appropriate reactive groups such as the reaction of diisocyanate macromers with dihydroxy macromers, and the reaction of diepoxy-containing macromers with dianhydride or diamine-containing macromers.

The biocompatible polymers for the magnetopolymer particles can be prepared in the form of dendrimers. The size, shape and properties of these dendrimers can be molecularly tailored to meet specialized end uses, such as a means for the delivery of high concentrations of biologically active agent per unit of polymer, controlled delivery, targeted delivery and/or multiple species delivery or use of biologically active agents. The dendrimeric polymers can be prepared according to methods known in the art, for example, Tomalia et al., U.S. Pat. No. 4,587,329, May 6, 1986 or Tomalia et al., U.S. Pat. No. 5,714,166, Feb. 3, 1998, herein incorporated by reference. Polyamine dendrimers may be prepared by reacting ammonia or an amine having a plurality of primary amine groups with N-substituted aziridine, such as N-tosyl or N-mesyl aziridine, to form a protected first generation polysulfonamide. The first generation polysulfonamide is then activated with acid, such as sulfuric, hydrochloric, trifluoroacetic, fluorosulfonic or chlorosulfonic acid, to form the first generation polyamine salt. The first generation polyamine salt can then be reacted further with N-protected aziridine to form the protected second generation polysulfonamide. The sequence can be repeated to produce higher generation polyamines. Polyamidoamines can be prepared by first reacting ammonia with methyl acrylate. The resulting agent is reacted with excess ethylenediamine to form a first generation adduct having three amidoamine moieties. This first generation adduct is then reacted with excess methyl acrylate to form a second generation adduct having terminal methyl ester moieties. The second generation adduct is then reacted with excess ethylenediamine to produce a polyamidoamine dendrimer having ordered, second generation dendritic branches with terminal amine moieties. Similar dendrimers containing amidoamine moieties can be made by using organic amines as the core agent, e.g., ethylenediamine which produces a tetra-branched dendrimer or diethylenetriamine which produces a penta-branched dendrimer.

The biocompatible polymers incorporated into the magnetic component particles for use in this embodiment may be, for example, biodegradable, bioresorbable, bioinert, and/or biostable. Bioresorbable hydrogel-forming polymers are generally naturally occurring polymers such as polysaccharides, examples of which include, but are not limited to, hyaluronic acid, starch, dextran, heparin, and chitosan; and proteins (and other polyamino acids), examples of which include but are not limited to gelatin, collagen, fibronectin, laminin, albumin and active peptide domains thereof. Matrices formed from these materials degrade under physiological conditions, generally via enzyme-mediated hydrolysis.

Bioresorbable matrix-forming polymers for the magnetopolymer particles are generally synthetic polymer prepared via condensation polymerization of one or more monomers. Matrix-forming polymers of this type include polylactide (PLA), polyglycolide (PGA), polylactide coglycolide (PLGA), polycaprolactone (PCL), as well as copolymers of these materials, polyanhydrides, and polyortho esters.

Biostable or bioinert hydrogel matrix-forming polymers for the magnetopolymer particles are generally synthetic or naturally occurring polymers which are soluble in water, matrices of which are hydrogels or water-containing gels. Examples of this type of polymer include polyvinylpyrrolidone (PVP), polyethylene glycol (PEG), polyethylene oxide (PEO), polyacrylamide (PAA), polyvinyl alcohol (PVA), and the like.

Biostable or bioinert matrix-forming polymers for the magnetopolymer particles are generally synthetic polymers formed from hydrophobic monomers such as methyl methacrylate, butyl methacrylate, dimethyl siloxanes, and the like. These polymer materials generally do not possess significant water solubility but can be formulated as neat liquids that form strong matrices upon activation. It is also possible to synthesize polymers that contain both hydrophilic and hydrophobic monomers.

The polymers used in the instant magnetic component particles of this embodiment can optionally provide a number of desirable functions or attributes. The polymers can be provided with water soluble regions, biodegradable regions, hydrophobic regions, as well as polymerizable regions.

Methods for forming the above various polymers and matrices are well know in the art. For example, various methods and materials are described in Chudzik et al., U.S. Pat. No. 6,410,044, issued Jun. 25, 2002; PCT Publication No. WO 93/16687; Jamiolkowski et al., U.S. Pat. No. 5,698,213, issued Dec. 16, 1997; Tomalia et al., U.S. Pat. No. 6,312,679, issued Nov. 6, 2001; Hubbell et al., U.S. Pat. No. 5,410,016, issued Apr. 25, 1995; Hubbell, et al., U.S. Pat. No. 5,529,914, issued Jun. 25, 1996; Rossling et al., U.S. Pat. No. 5,501,863, issued Mar. 26, 1996, which are all incorporated herein by reference.

The methods used to produce the magnetopolymer magnetic component particles result in particles that comprise one or more magnetic components, one or more biocompatible polymers and optionally one or more biologically active agents. Unlike previous compositions, the amount of iron oxide in the compositions of the present invention is limited and thus is present in a very small amount if there is any, for example, less than 5%. The magnetic components that are in the magnetic component particles to be used in the present embodiments are well-known materials with high magnetic susceptibility. Many of the magnetic components are commercially available in a variety of grades, including pharmaceutical grade.

Thus, a magnetopolymer for use in the present embodiment comprises up to 70% of a biocompatible polymer, 30% to 99% of a magnetic component, and from one part-per-billion to about 25% of a biologically active agent by mass. With compositions of greater than 70% polymer, the magnetic susceptibility of the particle is generally reduced beyond an optimal level for targeting biologically active agents in vivo.

Further description of ferrocarbon, ferroceramic and magnetopolymer magnetic component particles can be found at Rudge et al., U.S. application Ser. No. 09/673,297, filed on Oct. 13, 2000; Tapolsky et al., PCT Application No. PCT/US03/00489, filed on Jan. 7, 2003; and Rudge et al., U.S. Provisional Application No. 60/502,737, filed on Sep. 12, 2003, herein incorporated by reference.

The resulting magnetic component particles having optionally attached thereon one or more biologically active agents may be used alone or incorporated into a delivery system. Suitable delivery systems will be apparent to any person possessing ordinary skill in the art. The term “biologically active agent” is meant to include any agent having in vivo therapeutic properties, or having the ability to induce an in vivo response or effect, including the promotion of enhanced radiative effect.

A biologically active agent may be introduced to the raw magnetic component particles or to particles that have been processed, if desired, as is discussed more fully below.

When ready for use, one or more additional biologically active agents may be adsorbed or precipitated onto the magnetic component particles. The magnetic component particles, with the biologically active agent adsorbed, are introduced to the patient in a suspension of the magnetic component particles in a sterile diluent. In addition to absorbing deposited energy, the particles are also useful as a carrier for delivering one or more biologically active agents to targeted body sites guided by an external non-alternating magnetic field.

In one embodiment, the magnetic component particles used in the present embodiment can be associated with one or more biologically active agents for use in analytical or pharmaceutical applications. The combination of a magnetic component particle and a biologically active agent may be referred to as a “conjugate.” For example, the term “immunoconjugate” can refer to a conjugate comprising an antibody or antibody fragment and a magnetic component particle. Conjugates of a magnetic component particle and other molecules such as a label agent (e.g., a fluorophore), a binding ligand (e.g., a protein derivative), or a therapeutic agent (e.g., a therapeutic protein, toxin or organic molecule) can also be made by methods known in the art. For example, the conjugate is attached via a photocleavable bond, thus, upon exposure of the particle to light, the bond is broken and the conjugate is free to perform a desired function, at a highly specific time and place.

Conjugates can be prepared by covalently coupling one of the conjugate components to the other. Often coupling involves the use of a linker agent or a molecule that serves to join the conjugate components. A linker is typically chosen to provide a stable coupling between the two components. The greater the stability of the linkage between the components of a conjugate, the more useful and effective the conjugate. Depending upon a conjugate's use, a wide variety of conjugates may be prepared by coupling one conjugate component to another via a linker.

Alternatively, chelating structures can be employed to maintain the association of radionuclide biologically active agents to the magnetic component particles. Useful chelating structures include diethyltriaminepentaacetic acid (DTPA), structures based on the diamidodithiol (DADT) and triamidomonothiol (TAMT) backbones, and phosphinimine ligands. (See, Katti et al., U.S. Pat. No. 5,601,800, issued Feb. 11, 1997).

In one embodiment, additional biologically active agent targeting mechanisms can be optionally associated with the magnetic component particles. For example, an antibody, or fragment thereof, recognizing a specific ligand can be attached to the particles. Such immunoconjugates allow the selective delivery of biologically active agents to tumor cells. (See, e.g., Hermentin and Seiler, Behringer Insti. Mitl. 82:197-215 (1988); Gallego et al., Int. J. Cancer 33:7737-44 (1984); Arnon et al., Immunological Rev. 62:5-27 (1982)). For example, an antibody or antibody fragment recognizing a tumor antigen can be attached to a magnetic component particle. The antibody-containing particle can then be located at a tumor site by both a magnetic field and by antibody-ligand interactions. Alternatively, the methods and techniques described below need not be limited to simple attachment of the particle to tissue for localization, rather, the particle could be used as a means to retrieve information concerning the local environment of the particle.

Antibodies and antibody fragments, including monoclonal antibodies, anti-idiotype antibodies, and Fab, Fab′, F(ab′)₂ fragments or any other antibody fragments, that recognize a selected antigen can be obtained by screening antibodies and selecting those with high affinity. (See, U.S. Pat. No. RE 32,011, Wakabayashi et al., U.S. Pat. No. 4,902,614 issued Feb. 20, 1990, Frackelton et al., U.S. Pat. No. 4,543,439, issued Sep. 24, 1985, and Gillis, U.S. Pat. No. 4,411,993, issued Oct. 25, 1983; see also, Monoclonal Antibodies, Hybridomas: A New Dimension in Biological Analyses, Plenum Press, Kennett, McKearn, and Bechtol (eds.), 1980; Antibodies: A Laboratory Manual, Harlow and Lane (eds.), Cold Spring Harbor Laboratory Press, 1988)). Alternatively, antibodies or antibody fragments may also be produced and selected utilizing recombinant techniques. (See, e.g., Huse et al., Science 246:1275-1281 (1989); see also, Sastry et al., Proc. Natl. Acad. Sci. USA 86:5728-5732 (1989); Alting-Mees et al, Strategies in Molecular Biology 3:1-9 (1990)).

In addition, biologically active agents such as ligands recognized by receptors can be associated with a magnetic component particle. For example, neuraminic acid or sialyl Lewis X can be attached to a magnetic component particle. Such a ligand-containing particle can then be guided to a targeted site, such as an endothelial site, by both a non-alternating magnetic field and by ligand-selectin interactions. Such conjugates are suitable for the preparation of a medicament for treatment or prophylaxis of diseases in which bacterial or viral infections, inflammatory processes or metastasizing tumors are involved. Other biologically active agents include ligands, such as protein or synthetic molecules that are recognized by receptors can be associated with a magnetic component particle. In addition, one or more biologically active agents such as peptide, DNA and/or RNA recognition sequences can be associated with a magnetic component particle.

The association of the biologically active agent targeting mechanism can be by a covalent or ionic bond. Katti et al., U.S. Pat. No. 5,601,800, Feb. 11, 1997, describes several methods for attaching biologically active agents, such as diagnostic agents, contrast agents, receptor agents, and radionuclides to particles. Useful linkers and methods of use are described in, for example, King et al., U.S. Pat. No. 5,824,805, issued Oct. 20, 1998; Toepfer et al., U.S. Pat. No. 5,817,742, issued Oct. 6, 1998; Yatvin et al., U.S. Pat. No. 6,339,060, issued Jan. 15, 2002, herein incorporated by reference.

In one embodiment, the magnetic component particles comprise an additional biologically active therapeutic or diagnostic agent. A diagnostic and/or therapeutic amount of a biologically active agent attached to the magnetic component particles will be determined by any person having ordinary skill in the art as that amount necessary to effect diagnosis and/or treatment of a particular disease or condition, taking into account a variety of factors such as the patient's weight, age, and general health, and the nature and severity of the disease. Magnetic component particles may be administered such that the final concentration in the target volume is about 0.5 to about 50 mg/cc.

Generally, any useful diagnostic and/or therapeutic biologically active agent may be attached to the magnetic component particles for guided delivery to a targeted site. The term “biologically active” also includes agents used for diagnostic purposes and having no apparent physiological, therapeutic effect. Bifunctional agents having both diagnostic and therapeutic properties are also contemplated. Biologically active agents that can be precipitated, adsorbed, or labeled onto the magnetic component particles are, for example, but not limited to muscarinic receptor agonists and antagonists; anticholinesterase agents; catecholamines, sympathomimetic drugs, and andrenergic receptor antagonists; serotonin receptor agonists and antagonists; local and general anesthetics; anti-migraine agents such as ergotamine, caffeine, sumatriptan and the like; anti-epileptic agents; agents for the treatment of central nervous system degenerative disorders; opiod analgesics and antagonists; anti-inflammatory agents, including anti-asthmatic drugs; histamine and bradykinin antagonists, lipid-derived autocoids; nonsteroidal anti-inflammatory agents and anti-gout agents; anti-diuretics such as vassopressin peptides; inhibitors of the renin-angiotensin system such as angiotensin converting enzyme inhibitors; agents used in the treatment of myocardial ischemia, such as organic nitrates, Ca²⁺ channel antagonists, beta-adrenergic receptor antagonists, and antiplatelet/antithrombotic agents; anti-hypertensive agents such as diuretics, vasodilators, Ca²⁺ channel antagonists, beta-adrenergic receptor antagonists; cardiac glycosides such as digoxin, phosphodiesterase inhibitors; antiarrhythmic agents; anti-hyperlipoprotenimia agents; agents for the control of gastric acidity and treatment of peptic ulcers; agents affecting gastrointestinal water flux and motility; agents that cause contraction or relaxation of the uterus; anti-protozoal agents; anthelmintic agents; antimicrobial agents such as sulfonamides, quinolines, trimethoprim-sulfamethoxazole; beta-lactam antibiotics; aminoglycosides; tetracyclines; erythromycin and its derivatives; chloramphenicol, agents used in the chemotherapy of tuberculosis; Mycobacterium avium complex disease, and leprosy; anti-fungal agents; and anti-viral agents; anti-neoplastic agents such as alkylating agents, antimetabolites; natural products such as the vinca alkaloids, antibiotics (e.g., doxorubicin, bleomycin and the like); enzymes (e.g. L-asparaginase), biological response modifiers (such as interferon-alpha); platinum coordination compounds, anthracenedione and other miscellaneous agents; as well as hormones and antagonists (such as the estrogens, progestins, and the adrenocorticosteriods) and antibodies; immunomodulators including both immunosuppressive agents as well as immunostimulants; hematopoietic growth factors, anticoagulant, thrombolytic and antiplatelet agents; thyroid hormone, anti-thyroid agents, androgen receptor antagonists; adrenocortical steroids, insulin, oral hypoglycemic agents, agents affecting calcification and bone turnover as well as other therapeutic and diagnostic hormones, vitamins, minerals blood products biological response modifiers, diagnostic imaging agents, as well as paramagnetic and radioactive molecules or particles. Other biologically active substances may include, but are not limited to monoclonal or other antibodies, natural or synthetic genetic material and prodrugs.

As used herein, the term “genetic material” refers generally to nucleotides and polynucleotides, including nucleic acids, RNA and DNA of either natural or synthetic origin, including recombinant, sense and antisense RNA and DNA. Types of genetic material may include, for example, genes carried on expression vectors, such as plasmids, phagemids, cosmids, yeast artificial chromosomes, and defective (helper) viruses, antisense nucleic acids, both single and double stranded RNA and DNA and analogs thereof. Also included are proteins, peptides and other molecules formed by the expression of genetic material.

The magnetic component particles are such that the one or more biologically active agents can be associated with the particle, e.g., adsorbed, grafted, encapsulated, or linked to the particle. Various methods of labeling, adsorbing and/or precipitating biologically active agents are known in the art. The specific parameters used in these processes will depend upon the character and quality of the surface of the magnetic component particles, as well as that of the biologically active agent(s), and the properties of the solutions employed. A person having ordinary skill within the art easily can determine these parameters. The content of biologically active agent in the magnetic component particle is between about one part-per-billion to about 25% of the final particle mass. As used herein, “associated with” means that the biologically active agent can be physically encapsulated or entrapped within the particle, dispersed partially or fully throughout the particle, or attached or linked to the particle or any combination thereof, whereby the attachment or linkage is by means of covalent bonding, hydrogen bonding, adsorption, absorption chelation, metallic bonding, van der Walls forces or ionic bonding, or any combination thereof. The association of the biologically active agent(s) and the magnetic component particles(s) may optionally employ connectors and/or spacers to facilitate the preparation or use of the conjugates. Suitable connecting groups are groups which link a biologically active agent to the particle without significantly impairing the effectiveness of the biologically active agent or the effectiveness of any other carried material present in the particle. These connecting groups may be cleavable or non-cleavable and are typically used in order to avoid steric hindrance between the biologically active agent and the particle. Since the size, shape and functional group density of the particle can be rigorously controlled, there are many ways in which the biologically active agent can be associated with the particle. For example, (a) there can be covalent, coulombic, hydrophobic, or chelation type association between the biologically active agent(s) and entities, typically functional groups, located at or near the surface of the particle; (b) there can be covalent, coulombic, hydrophobic, or chelation type association between the biologically active agent(s) and moieties located within the interior of the particle; (c) the particle can be prepared to have an interior which is predominantly hollow allowing for physical entrapment of the biologically active agent within the interior (void volume), wherein the release of the biologically active agent can optionally be controlled by congesting the surface of the particle with diffusion controlling moieties, or (d) various combinations of the aforementioned phenomena can be employed.

Further depending on the characteristics of the biologically active agents to be introduced onto the magnetic component particles (for example, molecular weight, chemical structure, redox properties, and solubility), any person having ordinary skill in the art may easily identify an appropriate method for introduction of the desired biologically active agent(s). For instance, it is known that the reduction of perrhenate leads to insoluble rhenium oxides; thus, a redox reaction would be a good choice for labeling iron or iron-containing particles with rhenium oxides.

As another example, the magnetic component particles may be incubated with the biologically active agent in a medium, for example, water, buffer, or solvent. Preferably, the medium should not include agents that are likely to solubilize the magnetic component. Initially, the amount of incubation time may be determined in the feasible and reasonable range of about 5 to about 90 minutes, and preferably in the range of about 15 to about 60 minutes. The incubation temperature may be determined in accordance with the stability of the desired biologically active agents. The incubation times and temperatures may be adjusted to achieve the optimal attachment for a unique application.

Additional methods include the addition of solvent, such as ethanol, addition of salt or change of pH so as to induce precipitation, evaporation, or reduction of volume. Another method may include lowering the temperature of the solution in which a biologically active agent is present so as to induce precipitation or crystallization of the agent. Any person having ordinary skill in the art would be familiar with the appropriate methods involved in labeling, adsorption and/or precipitation and would be able to adjust the methods accordingly without undue experimentation.

Chemicals may be introduced to the process, for example, to alter the solubility of the biologically active agents, to induce precipitation (for instance, a redox reaction), or to facilitate deposition onto the magnetic component particles (for example, pH modification, or adjustment of the hydrophilicity-lipophilicity balance of the solution). These chemicals may be included in the solution containing the biologically active agent or introduced after the magnetic component or magnetic component-containing particles have been added. Time, temperature, and conditions of the incubation reaction, as well as use of additional excipients or chemical substances, may be adapted to the properties and characteristics of the biologically active agent(s) to be attached to the magnetic component particles.

The magnetic component particle surface may be optimized, for example, to enhance binding of biologically active agents where desired, to enhance bioavailability and targeting efficiency, and/or to increase surface area without change to the overall particle size, as described above.

Biologically active agents such as radioisotopes are chemical agents or elements that emit alpha, beta or gamma radiation and that are useful for diagnostic and/or therapeutic purposes. One factor used in selecting an appropriate radioisotope is that the half-life be long enough so that it is still detectable or therapeutic at the time of maximum uptake by the target, but short enough so that deleterious radiation with respect to the patient is minimized. Selection of an appropriate radioisotope would be readily apparent to one having ordinary skill in the art. Generally, alpha and beta radiation are considered useful for local therapy. Examples of useful agents include, but are not limited to ³²P, ⁵⁵Co, ⁵⁶Co, ⁵⁷Ni, ¹⁸⁶Re, ¹⁸⁸Re, ¹²³I, ¹²⁵I, ¹³¹I, ⁹⁰Y, ¹⁶⁶Ho, ¹⁵³Sm, ¹⁴³Pr, ¹⁴⁹Tb, ¹⁶¹Tb, ¹¹¹In, ⁷⁷Br, ²¹⁴Bi, ²¹³Bi, ²²⁴Ra, ²¹⁰Po, ^{195m}Pt, ¹⁶⁵Dy, ¹⁰⁹Pd, ^{117m}Sn, ^{123m}Te, ¹⁰³Pd, ¹⁷⁷Lu, and ²¹¹At. The radioisotope generally exists as a radical within a salt, with the notable exception of the Iodine's. The useful diagnostic and therapeutic radioisotopes may be used alone or in combination.

For in vivo diagnostic imaging, for assessing the location of the particles, the type of detection instrument available is a major factor in selecting a given radioisotope. The radioisotope chosen must have a type of decay that is detectable for a given type of instrument. Generally, gamma radiation is required. Still another important factor in selecting a radioisotope is that the half-life be long enough so that it is still detectable at the time of maximum uptake by the target, but short enough so that deleterious radiation with respect to the host is minimized. Selection of an appropriate radioisotope would be readily apparent to one having average skill in the art. Radioisotopes that may be employed include, but are not limited to ^{99m}Tc, ¹⁴²Pr, ¹⁶¹Th, ¹⁸⁶Re, and ¹⁸⁸Re. Additionally, typical examples of other diagnostically useful agents are metallic ions including, but not limited to ¹¹¹In, ⁹⁷Ru, ⁶⁷Ga, ⁶⁸Ga, ⁷²As, ⁹⁵Zr, and ²⁰¹Tl. Furthermore, paramagnetic elements that are particularly useful in magnetic resonance imaging and electron spin resonance techniques include, but are not limited to ¹⁵⁷Gd, ¹⁶²Dy, ⁵¹Cr, and ⁵⁹Fe. Where isotopes correspond to the magnetic composition, for instance with Fe, Ni and Co, the isotope may comprise part of the magnetic composition of the magnetic component particles.

In one embodiment, the deposited energy is applied by the use of an external magnetic field. A review of the use of RF fields on magnetically susceptible particles can be found in Moroz et al., Int. J. Hyperthermia, 18:267-284 (2002), herein incorporated in its entirety by reference.

In another embodiment, the RF magnetic field may be applied from an internal magnetic field, that is, the source of the field is internal with respect to the exterior surface of the skin.

Application of the deposited energy may be reapplied to the same magnetic component particles for as long as they persist in the targeted site. Such deposited energy may be applied with capacitive heating devices, such as instruments like the Thermotron RF-8, Yamamoto Vinyter Co., Osaka, or the RF2000 Generator with a 2.0 cm probe (Boston Scientific, Natick, Mass.) for RF Ablation, for example. Deposited energy supplies are well known in the art and commercially available.

In one embodiment, the deposited energy is applied by a radiofrequency (RF) capacitive device. One embodiment of such a process, both for in vitro and in vivo applications, is disclosed for a different type of particle in Shinkai et al., Jpn. J. Cancer Res. 90:699-704 (1999), herein incorporated in its entirety by reference. Shinkai et al. show that magnetite particles can be injected into a patient and a Thermotron RF-8 can be used to generate an RF field; the field is applied, via electrodes, to the subject. In the present embodiment, magnetic component particles are used since the iron oxides used in Shinkai et al. are not defined as magnetic compositions for the present embodiment. As such, the duration of the application of the magnetic field to the subject will be reduced for the current embodiment. Likewise, before the application of the RF field, a non-alternating magnetic field is first used to guide the particles to the targeted site, again resulting in an even greater reduction in the amount of heating required to destroy a particular tissue sample. These differences also apply for the in vivo applications. For in vivo applications, it is often important to make sure that the temperature of the surrounding tissue does not rise to too high a temperature, resulting in undesired tissue damage. In such cases, it is often desirable to increase the power of the RF field in steps while monitoring the resulting temperature. It is possible to achieve temperatures of 43° C. at the targeted site of the magnetic component particles, while the surrounding tissues are still under 39.8° C. This is important since it is sufficient to cause cell death at the targeted site, without destroying the neighboring healthy cells.

In one embodiment, the RF energy is applied by placing the patient inside an alternating magnetic field. First, magnetic component particles are magnetically guided to the targeted site, for example an organ, tissue or tumor by use of a non-alternating magnetic field. The subject is then placed into a device that can generate an alternating magnetic field, such as a multiturn magnetic coil and the magnetic field set to 340 Oe, alternating at 20 kHz for 5 minutes. The descriptions of such alternating magnetic fields can be found in Hilger et al., Investigative Radiology, 37:580-586 (2002); or Moroz et al., Journal of Surgical Research, 105:209-214 (2002), both of which are herein incorporated in their entireties by reference. In the present embodiment, the particles used are not the iron oxide particles described in Moroz et al; additionally, the particles of the present invention can be, and are then, magnetically guided to a targeted site, following infusion of the particles. This latter step has many advantages, not the least of which is the reduction in the need to clamp any arteries of the patient before applying a magnetic field to the patient.

There are many methods that one can use to determine possible heating rates of tissues in order to theoretically determine the temperature of the targeted site. One possible method is described by Moroz et al., Journal of Surgical Research, 105:209-214 (2002) involving the determination of a linear regression equation in the case of a tissue sample of pig's kidney. The heating rate (HR, in degrees per minute) can be determined as a function of the renal tissue iron concentration (mg/g).

HR=0.20×Fe+0.19

Similarly, one of skill in the art could determine other appropriate formulae for the heating rate of other tissues.

In an alternative embodiment, the deposited energy applied is electrical energy, and is enhanced by the increased electrical conductivity of the magnetically guided magnetic component particles in the targeted site. The effect would be similar to the enhancement observed when NaCl is applied to the targeted site. A description of the application of NaCl is described in Goldberg et al., Radiology, 219:157-165 (2001), herein incorporated in its entirety by reference. Advantageously, the magnetic component particles are extravasated and thus immobilized in the targeted site, while the NaCl is flushed from the targeted site by the flow of blood.

In another embodiment, the deposited energy applied to the magnetic component particles is a RF field administered via a RF probe. There are many RF probes known in the art such as the one that can be found in Edwards et al., U.S. Pat. No. 6,471,698, issued Oct. 29, 2002, herein incorporated in its entirety by reference.

In another embodiment, the deposited energy applied is in the form of radiation or nuclear energy. In one embodiment, the particles that are magnetically guided to a targeted site act as a shield to protect other organs from the deposited energy that is being applied. In another embodiment, the thermal or nuclear cross-section of the particles are optimized so as to capture the energy being applied to the particles and result in an increase in heat of the particles, at a faster rate than the surrounding tissues. In one embodiment, the absorption of the energy may result in the release of a biologically active agent from the particle, for example as free radicals.

Thus, in one embodiment, the deposited energy in the form of radiation is applied in a frequency selective manner, as described in Mills, U.S. Pat. No. 4,815,447, issued Mar. 28, 1989, herein incorporated in its entirety by reference. Briefly, energy absorbed at particular frequencies, so called Mossbauer absorption frequencies, are converted into and remitted as Auger electrons. Auger electrons provide intranuclear radiation resulting in lethal double strand breaks in the DNA of the surrounding cells. Thus radiation that is relatively harmless to the surrounding tissues passes through them and into the magnetic component particles, whereupon the energy reemerges in a cell lethal form. In this embodiment, rather than trying to determine the frequency of radiation at which to bombard a particular tissue type, the use of the present magnetic component particles allows one to already know the required frequency. Likewise, the use of the magnetic component particles allows one to localize the effect, as well as target organs that might otherwise be too risky to treat by conventional nuclear radiation.

Also, in another embodiment, the deposited energy is gamma radiation. In another embodiment, the deposited energy is nuclear energy in the form of beta radiation. In another embodiment the deposited energy is radiation in the form of alpha radiation. In a one embodiment, the radiation is from neutrons. In one embodiment, the neutrons are used for neutron capture therapy. This therapy involves the application of neutrons to tissue that is doped with either Boron or Gadolinium. The result is a fission reaction, where the fission products remain very localized (within 5 to 10 microns of reaction size). When boron is used, lithium, hydrogen, nitrogen, and alpha and gamma rays are produced, damaging the local cells. When gadolinium is used, Auger electrons and gamma rays damage the local cells. The use and production of such molecules can be found in Perkins et al., U.S. Pat. No. 6,627,176, issued Sep. 30, 2003, describing possible metal complexes that can be used and methods for connecting the complex to other agents, herein incorporated in its entirety by reference. Additionally, alternative methods for the application of such molecules are described in Hawthorne, U.S. Pat. No. 6,517,808, issued Feb. 11, 2003, herein incorporated in its entirety by reference. In order for this therapy to be effective, sufficient amounts of the particles must be localized in a tumor to generate the required density of particles. This level has been variously estimated to be approximately 10-50 micrograms ¹⁰B/gm tumor. Furthermore, the concentration of ¹⁰B in normal tissue and blood should be limited and preferably be less than the concentration in the targeted site tumor in order to minimize damage to healthy cells and blood vessels. See, H. Hatanaka, Boron-Neutron Capture Therapy for Tumors; Nishimura Co., Ltd. p. 1-16 (1986) herein incorporated in its entirety by reference. One major advantage of the current embodiment is that the magnetic guidance of the magnetic component particles to targeted sites reduces both of these dangerous side effects of neutron capture therapy.

In one embodiment, the source of the deposited energy in the form of radiation or nuclear energy is in the form of heavy particles. In another embodiment, the source of the radiation or nuclear energy is from a particle beam. As will be appreciated by one of skill in the art, the actual source of the energy is not critical, so long as the energy can reach the particles.

In another embodiment, the deposited energy can be administered simultaneously with other forms of energy. There is no theoretical limit on the types or numbers of treatments that can be administered at once, so long as they do not interfere detrimentally with each other. In one embodiment, ultrasound and photon radiation are applied at the same time. In an alternative embodiment, photon radiation and microwaves are applied at the same time. Straube et al., Int. J. Hyperthermia 17:48-62, (2001), herein incorporated in its entirety by reference, discloses how to apply both of the prior two forms to a patient without any magnetic component particles in his system.

In another embodiment, the deposited energy is applied in conjunction with an additional treatment. For example, the particles of the present embodiment may be administered (introduced) to a patient and magnetically guided to a targeted site. Additionally a biologically active agent, such as doxorubicin, can also be administered to a patient, and then the deposited energy can be applied to the magnetic component particles. The background for methods for doing this can be found in Goldberg et al., Radiology, 220:420-427 (2001), herein incorporated in its entirety by reference. Goldberg et al. does not teach the use of magnetic component particles. In one embodiment, the biologically active agent to be used for chemotherapy is attached to the particles themselves, thus allowing for the localization of both types of treatments by the application of the guiding magnetic field. In an alternative embodiment, the molecule for chemotherapy that is attached to the particle is only chemically active once the deposited energy has been applied to the particles, thus allowing for the magnetic guidance of the particles and the treatment, without any impact on the surrounding tissues.

In another embodiment, the deposited energy can be administered in the form of microwaves. While microwaves usually result in undesired heating of surface tissues, a microwave probe can be used to reduce any such heating. One such probe is disclosed in Yerushalmi, U.S. Pat. No. 4,601,296, issued Jul. 22, 1986, herein incorporated in its entirety by reference. As will be appreciated by one of skill in the art, the placement of a microwave probe, surrounded by a cooled shell, into the patient will reduce any damage that occurs due to the microwaves heating the surrounding tissues. Likewise, the microwaves will be converted to heat more readily by the magnetic component particles than by the surrounding tissue, thus a low application, over a period long enough to allow for fluid exchange in the local environment, will create a situation where the energy can be transferred from the probe, without excessive damage to the local, healthy, tissue.

In one embodiment, traditional radio frequency (RF) ablation techniques can be applied with the magnetically guided magnetic component particles of the present invention. While RF ablation works by passing an electrical current from at least one, and usually between two, electrodes, the use of such a RF ablation technique, in conjunction with the present particles, should help to localize and direct the current that is passed between the electrodes. That is, if the magnetic component particles are more conductive to current than the surrounding tissue, the current to be passed during RF ablation will be more concentrated in the targeted sites between the electrodes that have the particles. This allows for the targeted sites that are between the electrodes and doped with the particles, to be treated with current at a greater level than the surrounding tissue, thus reducing healthy tissue damage. As will be appreciated by one of skill in the art, the combination of magnetic component particles magnetically guided to targeted sites, as described herein, and an RF ablation technique has advantages over RF ablation techniques alone. While a traditional RF ablation technique suffers from inefficiency due to heat loss from the surrounding tissue due to blood circulation, the ability to magnetically guide and extravasate the particles of the current invention allows for some of the heated particles to remain in the desired location over a prolonged period of time. Another advantage of the current embodiment is that the heating rate of the particles can be greater than the surrounding tissue, thus allowing for a shorter treatment time, which also allows for less damage to surrounding healthy tissue.

In one embodiment, the magnetic field used to guide the magnetic component particles to the targeted site is maintained during treatment, thus keeping the particles in a particular place throughout the treatment. In an alternative embodiment, the particles, once magnetically guided to a specific location, are allowed to associate with the surrounding tissue and remain in place through those associations, for example by bonds or physical entrapments. In an alternative embodiment, the magnetic component particles comprise an additional biologically active agent element, such as an antibody directed to a marker on the targeted site, and can associate with the tissue in that manner. In another embodiment, the magnetic field is used to embed the particles into the targeted site, thus increasing heat transfer and immobilizing the particles.

In one embodiment, the deposited energy applied is in the form of ultrasound. In one embodiment, the ultrasound is in the form of high-intensity focused ultrasound (HIFU). The magnetic component particles of the present invention are again magnetically guided to a targeted site in the tissue, whereupon the particles are bombarded with HIFU, which results in an increase in the temperature of the particles, due to their absorption of the energy. The ability of the particles to absorb the energy will be determined by their acoustic characteristics. In one embodiment, the acoustic frequency of the magnetic component particles is different from that of the tissues through which the HIFU beam passes, thus the particle can act as a “sounding board” to create heat at a desired location, without the creation of heat from the surrounding tissues. For a description of HIFU, see Ahmed and Goldberg, J. Vasc. Interv. Radiol., 13:S231-S243 (2002), herein incorporated in its entirety by reference.

In one embodiment, the deposited energy applied is in the form of a laser. The laser may be applied completely externally, thus risking some transfer of the beam through healthy tissue. Alternatively, the laser may be applied via a fiber optic and thus the proximity of the laser source to the magnetic component particles may be increased. For a description of such a laser, see Ahmed and Goldberg, J. Vasc. Interv. Radiol., 13:S231-S243 (2002), herein incorporated in its entirety by reference. The application of the laser in the current invention will be to the magnetic component particles and the targeted tissue, rather than simply to the tissue in general. Thus, the optical properties of the particles will dictate how this process is best employed. In one embodiment, magnetic component particles that absorb light and emit heat, or emit a wavelength of cell damaging light. In another embodiment, magnetic component particles that redistribute light, such as a prism like device, may be desired, in order to apply the damaging effects of the laser to a larger area of the tissue.

In an alternative embodiment, the deposited energy is a form of light. In one embodiment, as in the laser embodiment above, the light itself may be damaging or may simply heat the magnetic component particles of the present invention.

In an embodiment, the deposited energy is in the form of light, and the magnetic component particles contain an agent for photodynamic therapy (PDT). Methods and agents for PDT are well known in the art. PDT is a method of treating a diseased tissue of a patient. Typically, the surgical procedure involves administering a photodynamic agent to a patient, such as via an intravenous injection, and then irradiating the target diseased tissue with a separate light source. The photodynamic agent, following irradiation with light, emits reactive oxygen species, such as singlet oxygen, which disrupts the surrounding cellular tissue. Examples of the technique can be found in: Love et al., U.S. Pat. No. 6,630,128, issued Oct. 7, 2003; Crean et al., U.S. Pat. No. 6,586,419, issued Jul. 1, 2003; Miller et al., U.S. Pat. No. 6,610,679, issued Aug. 26, 2003; Levy et al., U.S. Pat. No. 4,883,790, issued Nov. 28, 1989; Levy et al., U.S. Pat. No. 4,920,143 issued Apr. 24, 1990; Levy et al., U.S. Pat. No. 5,095,030, issued Mar. 10, 1992; and Levy et al., U.S. Pat. No. 5,171,749, issued Dec. 15, 1992; Levy et al., U.S. Pat. No. 6,100,290, issued Aug. 8, 2000, Obochi et al., U.S. Pat. No. 6,364,907, issued Apr. 2, 2002, all herein expressly incorporated in their entireties by reference.

Normally, the accumulation, by a cell, of photoactive biologically active agents, such as Photophrin® (QLT Inc., Vancouver, B.C.), is necessary for PDT. Using the instant method to magnetically guide the Photophrin® to the targeted site in order to have the photoactivation of the Photophrin® destroy the cells of the targeted site. One advantage of this combination of techniques is that by being able to magnetically guide the particles that are combined with Photophrin® directly to the targeted site, for instance, a tumor, one does not have to wait the 40 to 50 hours that one would normally be required in PDT. As will be appreciated by one of skill in the art, Photophrin® is merely an example of one type of photoactive or photodynamic biologically active agent that can be used and should not limit the present embodiment.

In an alternative embodiment, the deposited energy is in a form of light, and the magnetic component particles contain biologically active agent groups that are photoactive, thus the presence of light cleaves a bond or alters the chemical properties of this agent that has been magnetically guided to the targeted site by the particle. This allows the particles to be magnetically guided to the targeted site, with their attached photoactive biologically active agents, without the agents becoming active before they have been targeted. Likewise, the associated agents on the particles may be a lethal factor that is to be delivered to a targeted site, for example a tumor. If the biologically active agents are connected to the magnetic component particles with photocleavable bonds, then one can control their delivery in a highly specific manner. Photocleavable bonds and photoactive agents in general are well known in the art and the selection of the appropriate photoactive molecule is routine for one of skill in the art. For a list of examples of photoactive agents, see the Handbook of Fluorescent Probes and Research Products, by Molecular Probes, in particular, the chapters concerning photoactive and photoreactive reagents, especially chapter 5 of the ninth edition, (Molecular Probes; Eugene, Oreg.), herein incorporated in its entirety by reference.

In an alternative embodiment, the deposited energy applied to the magnetic component particles is in the form of a lowered temperature. In one embodiment, the mere presence of the particles in the body will alter the rate of heat exchange for the tissues that contain the particles. While application of extreme cold will usually kill all of the tissues (i.e. the organism), not just the particle embedded tissues, a small reduction in temperature, applied to a targeted site to which magnetic component particles have been magnetically guided, will allow for heat to be exchanged through the targeted site faster than through the neighboring, water-based tissues. Thus, the presence of these particles will allow one to effectively freeze the targeted site, before the surrounding tissue is frozen. The reduction in temperature may be applied to the entire body. More preferably, it will be applied to an isolated area with a heat exchange device. In one embodiment, the device is a peltier device. In another embodiment, the peltier device can be implanted into the patient in order to achieve more efficient uptake of heat from the magnetic component particle embedded tissue. In another embodiment, the magnetic component particles used in the present invention are combined with a process similar to SEEDNET™ (Galil Medical, Westbury, N.Y.) such as that described in Schatzberger et al., U.S. Pat. No. 6,142,991, issued Nov. 7, 2000, herein incorporated in its entirety by reference. Briefly, a series of ultra-fine probes are inserted into the targeted site, where the probes can cause local freezing. The presence or absence of the particles of the present embodiment can be used to help refine the sections of tissue that are cooled. The magnetic component particles may help conduct heat away, and thus cool sections of tissue. On advantage to doping the tissue with the particles is that the particles, unlike the ice created by the freezing technique, will be able to achieve a temperature below 0° C., thus improving the inefficiencies in the SEEDNET™ system. Alternatively, the particles could be used to insulate the tissue from the cold treatment, by their inherent characteristics, or by the application of another energy deposition.

In another embodiment, the deposition of energy has an enhanced or synergistic effect on the associated biologically active agents or properties of the magnetic component particles once magnetically guided to a target site. At a simple level, this may involve a magnetic field heating a magnetic component particle. In turn, the magnetic component particle will heat the surrounding environment. This heating may serve to destroy local tissues directly, but the heating may also serve to increase the functionality of any associated biologically active agents. This increase in temperature may increase the rate of reaction of a basic chemical reaction, or it may increase the rate of catalysis of an enzyme that is associated with the magnetic component particle. Alternatively, the deposition of energy may result in the production of other elements that produce a synergistic effect with either the magnetic component particle or the biologically active agents associated with the magnetic component particles. For example, the deposition of energy may also result in the production of free radicals, as well as heat, both of which may kill neighboring cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The terms “a” and “one” are both meant to be interpreted as “one or more” and “at least one.” All publications, patent applications, patents and other references mentioned herein are incorporated by reference in their entirety. In case of conflict, the present specification, including definitions, will control. In addition, the materials, methods and examples are illustrative only and not intended to be limiting.

### EXAMPLE 1

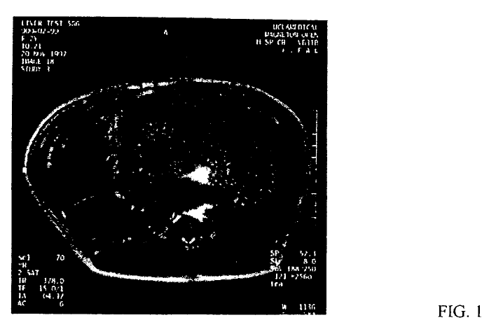

Magnetic component particles such as those described may be used to target tissues very uniformly. For example, when particles are infused intra-arterially to a hepatic tumor then magnetically guided to the target site, very uniform distribution is achieved. This is demonstrated in FIG. 1, which shows a magnetic resonance image of such a tumor after infusion of these particles under the influence of a magnetic field. The figure shows nearly homogeneous distribution of the particles within the region of interest, as evidenced by the negative artifact in the image. Since the particles are delivered via the microvasculature, the space between particles is on the order of individual cells. Current technologies deliver magnetic component particles to the periphery of the tumor, as they are larger than the microvasculature, or are implanted at distances at least one millimeter (1000 μm apart). In these cases, it is advantageous to have uniform distribution of the particles, as studies have shown the effectiveness of the treatment is often limited by the region of least effective energy deposition.

### EXAMPLE 2

Magnetic component particles, such as those described, may be used at varying concentrations in the targeted tissue. The concentration of the particles may affect the efficiency of the energy deposition, or the uniformity of the energy deposition. It is important that the deposition of the energy be tunable, as too much or too little energy deposition are either harmful or ineffective. In 33 patients with primary hepatocellular carcinoma, treated with particles such as those described, particles were infused intra-arterially such that then magnetically guided to the target site the resulting concentration in the targeted site would range from 0.6 to 31 mg/cc.

### EXAMPLE 3

The following table demonstrates some of the physical processes by which energy deposition is enhanced by virtue of the distribution of magnetic component particles in the targeted site. These examples are not meant to be limiting, as additional enhancements are also likely.

| | | Enhancement by virtue of |
|---|---|---|
| Deposited Energy | Mode of Therapy | described particles |
| Electrical | Heating of tissue to induce | The particles increase |
| | coagulation or tissue damage | the electrical |
| | | conductivity of the |
| | | tissue, increasing the |
| | | current at constant |
| | | voltage, thereby |
| | | increasing the heating, |
| | | W = I²R |
| | | The particles increase |
| | | the thermal conductivity |
| | | of the tissue, thereby |
| | | directing the energy |
| | | flow to the region of |
| | | interest, rather than |
| | | allowing diffuse |
| | | penetration of the heat. |
| Magnetic | Heating of tissue to induce | The particles heat inductively |
| | coagulation or apoptosis | in the alternating magnetic |
| | | field, and distribute heat to |
| | | the surrounding tissue. Since |
| | | the particles are capable of |
| | | being distributed at the |
| | | cellular level, the distribution |
| | | of heat is uniform on the |
| | | scale of 10 to 20 microns. |
| Nuclear (gamma, beta, alpha, | Cleavage of or damage to | The efficiency of |
| neutron, heavy particle, | cellular DNA, generation of | capture of nuclear |
| particle beam) | free radicals, disruption of | radiation is related to |
| | cellular membranes | the density of the |
| | | medium. Particles could |
| | | protect antecedent |
| | | tissue by absorbing |
| | | radiation. The result of |
| | | particles capturing the |
| | | radiation would be |
| | | generation of free |
| | | radicals or molecules, |
| | | such as Fe⁺² with large |
| | | electronegative |
| | | potential |
| | | A component of the |
| | | particle could be |
| | | designed to be highly |
| | | efficient for the capture |
| | | of radiation (called the |
| | | “cross section” for a |
| | | particular type of |
| | | radiation) and to emit a |
| | | particularly toxic (or |
| | | efficacious) molecule |
| | | (see neutron capture |
| | | therapy). |
| Photon | Activation of a prodrug, free | See above |
| | radical generation, heat | |
| | generation | |
| Cryogenic | “Burning” of tissue through | Increased thermal |
| | freezing | conductivity of tissue, |
| | | direction of freezing from the |
| | | targeted tissue margins |
| | | inward. |

### EXAMPLE 4

This example demonstrates a method for determining the heating ability of a particular set of magnetic component particles, for a particular type of tissue; in this example, the tissue to be simulated is liver. Liver and egg whites have similar thermal conductivity and electrical conductivity. An egg's albumin coagulates at 60° C. and generates a visible, measurable opaque region, so the heating effect could easily be recorded using digital imaging acquisition equipment. While 60° C. is greater than the relevant 42-43° C. desired for in vivo use, this example is only performed to obtain a heating rate, which will be extrapolated to the lower temperature ranges. Alternatively, a thermometer could be included in the egg whites in order to observe the lower temperature ranges. The particles of the present embodiment are added to the egg whites and a 2.0 cm RITA Medical RF probe (Mountainview, Calif.) is deployed in the middle of the sample. RF energy will be delivered at 50 W for 15 min. or until maximum coagulation is achieved. Temperature measurements will be made at various locations from the electrode source, in order to determine the effective temperature at locations far from the source. Various frequencies can be tried, and various concentrations as well. Ideally a range of both will be tried, starting with 500 kHz for the frequency, and 25 mg/ml, 10 mg/ml, 5.0 mg/ml, 1.0 mg/ml, and 0.5 mg/ml for the concentration. Additionally, by selectively placing the particles between the electrodes of the RF probe and taking the temperature of both the area with the particles and the area without the particles, one is able to determine the temperature of the targeted tissue with the particles and the temperature of the tissue without the particles. Thus one can determine the effectiveness of localizing the particles in a RF ablation experiment.

### EXAMPLE 5

### Magnetic Susceptibility

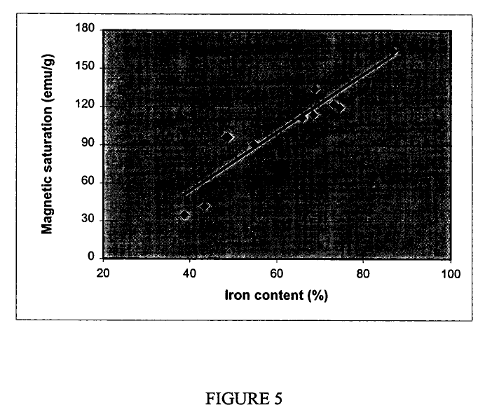

Example 5 contrasts the magnetic susceptibility of the magnetic component particles with those of magnetite based particles. Magnetic saturation vs. the magnetic component content of these particles is shown in FIG. 5. The magnetic saturation increases with the magnetic component content. The greater the magnetic saturation, the greater the degree of the magnetic attraction (capture), and the deeper the particles can be targeted in vivo.

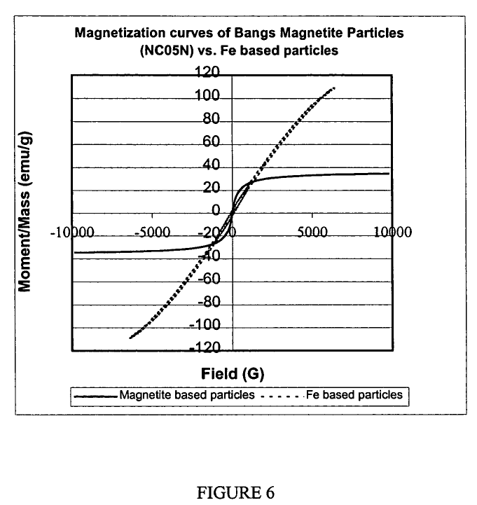

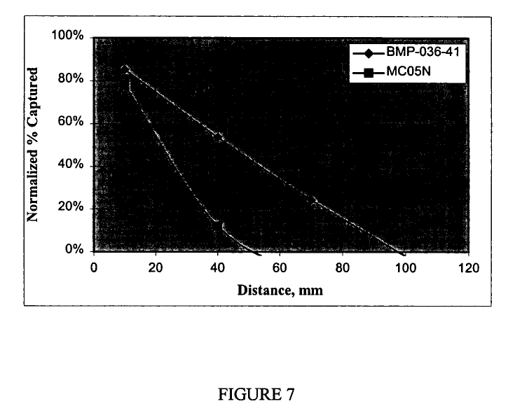

FIG. 6 illustrates the magnetization curves of Bang's magnetite particles (NC05N) vs. Fe-based magnetic component particles. The PLGA/Fe magnetic component particles not only have a much higher magnetic saturation, they also have a different characteristic magnetization hysteresis curve. As shown in FIG. 7, a PLGA/Fe magnetic component particle preparation with 50.6% Fe has a magnetic saturation greater than 108 emu/g, while a generic magnetite based particle (Bangs Magnetite Particles, catalog MC05N, Poly(styrene-divinylbenzene 6% /V—COOH) Magnetite 52.4%, Inv. # L951211D, Bangs Lot# 1975), Bangs Laboratories, Inc., Fishers, Ind. has a saturation magnetization of only 34.7 emu/g. The theoretical saturation magnetization for magnetite and metallic iron are 92 and 218 emu/g, respectively (Craik, D., Magnetism Principles and Applications. Wiley and Sons, 1995). The labeled magnetite content of the particles is 52.4%, so a saturation magnetization of approximately 50 emu/g was expected. This shows that only 70% of the expected magnetic properties are retained by magnetite when it is dispersed as a fine powder and covered by polymer. In a like manner, the magnetic component particle, which is 50.6% Fe by weight, would be expected to have a saturation of 109 emu/g. Therefore, the magnetic component particle retains approximately 100% of the expected magnetic saturation. This shows that while both particle types retain their magnetic properties, the magnetic component particle is better at retaining these properties when formed into a finely dispersed microsphere, and is unexpectedly superior to an iron oxide-based particle in terms of its magnetic properties. A large advantage of this embodiment is that the coercivity, which is related to the amount of inductive heating that would be expected in an alternating magnetic field, is more than six times higher for magnetocarbon magnetic component particles (1.2 emu/g) than for simple magnetic based (0.19 emu/g) particles.

### EXAMPLE 6

### Magnetic Capture

This example demonstrates the importance of using a magnetic component, such as metallic iron, instead of iron oxide to achieve efficient magnetic capture and targeting. A magnetic component particle comprising about 50% metallic iron was investigated for its capture by a magnetic field in a flow field. Some commercially available magnetic particles (MC05N, ˜1 μm in size and 60% of magnetite by weight from Bangs Laboratories (Fisher, Ind.)) were used as reference. FIG. 7 illustrates the percent captured based on the number of particles vs. distance between the magnet and particles. The magnetic component particle, BMP-036-41, showed much higher magnetic capture efficiency. The magnetic capture for Bangs particles (MC05N) diminished quickly with the increase of distance from the magnet.

What is claimed is:

1. A method of radiative therapy comprising:
a) introducing more than one magnetic component particle into a patient;
b) magnetically guiding with a non-alternating magnetic field the magnetic component particle to a targeted site; and
d) depositing energy at the targeted site.

2. The method of claim 1, wherein the magnetic component particle comprises a metal with more than 75% metallic iron.

3. The method of claim 1, wherein iron in the magnetic component particle is less than 10% iron oxide.

4. The method of claim 1, wherein the magnetic component particles comprises a magnetosorptive particle.

5. The method of claim 4, wherein the magnetosorptive particle has a weight ratio of magnetic component:sorbent in the range from about 95:5 to about 50:50.

6. The method of claim 4, wherein the magnetosorptive composition comprises magnetocarbon particles.

7. The method of claim 6, wherein the magnetocarbon particles comprise at least one type of activated carbon, selected from the group consisting of type A, type B, type E, type K, and type KB.

8. The method of claim 6, wherein the magnetocarbon particles further comprise one or more biologically active agents.

9. The method of claim 8, wherein the one or more biologically active agents are selected from the group consisting of antibiotics, antifungals and antineoplastic agents.

10. The method of claim 4, wherein the magnetosorptive composition comprises magnetoceramic particles.

11. The method of claim 10, wherein the ceramic is selected from the group consisting of a natural porous adsorptive material and a synthetic porous adsorptive material.

12. The method of claim 10, wherein the ceramic is selected from the group consisting of hydroxyapatite, silicas and chemically modified silicas.

13. The method of claim 10, wherein the magnetoceramic particles further comprise one or more biologically active agents.

14. The method of claim 13, wherein the one or more biologically active agents are chosen from the group consisting of antifungals, antineoplastics and antibiotics.

15. The method of claim 1, wherein the magnetic component particles are magnetopolymer particles.

16. The method of claim 15, wherein the polymeric components are biodegradable polymers.

17. The method of claim 16, wherein the polymeric component is PLGA.

18. The method of claim 15, wherein the magnetopolymer particles further comprise one or more biologically active agents.

19. The method of claim 15, wherein the one or more biologically active agents are chosen from the group consisting of antifungal, antineoplasic and antibiotics.

20. The method of claim 1, wherein the magnetic component particles are processed.

21. The method of claim 20, wherein the process is selected from the group consisting of gas phase treatment, mechanical milling, spray drying, heating, cooling, annealing, and plastic deformation.

22. The method of claim 1, where the magnetic component particles further comprise one or more biologically active agents that are one or more isotopes.

23. The method of claim 1, wherein one or more biologically active bifunctional agent are attached to the particles.

24. The method of claim 1, wherein the size of the particles is less than 5 cm.

25. The method of claim 24, wherein the average size of the particles in the magnetic composition is between approximately 0.1 microns to approximately 20 microns.

26. The method of claim 24, wherein the average size of the particle is from between about 0.5 to about 5 microns.

27. The method of claim 1, wherein the magnetic component particles are introduced with a delivery vehicle.

28. The method of claim 1, wherein the magnetic component particles are introduced with one or more excipients.

29. The method of claim 1, wherein the particles are introduced by a method selected from the group consisting of injection, infusion, implantation, and ingestion.

30. The method of claim 1, wherein the targeted site is selected from the group consisting of tumors, infections, aneurysms, abscesses, viral growths, and other focal points of disease.

31. The method of claim 1, also comprising the introduction of an embolic agent.

32. The method of claim 32, wherein the embolic agent is a second batch of magnetic component particles, wherein the larger particles are used as the embolic agent.

33. The method of claim 1, wherein the deposited energy is applied for an amount of time effective to obtain a therapeutic effect.

34. The method of claim 1, wherein protective compositions are used in the area surrounding the target.

35. The method of claim 1, wherein the deposited energy is applied with a RF capacitive heating system.

36. The method of claim 1, wherein the deposited energy is tunable.

37. The method of claim 1, wherein the deposited energy is electrical.

38. The method of claim 1, wherein the deposited energy is alternating magnetic energy.

39. The method of claim 1, wherein the deposited energy is nuclear.

40. The method of claim 39, wherein the nuclear energy is from gamma particles.

41. The method of claim 39, wherein the nuclear energy is from beta particles.

42. The method of claim 39, wherein the nuclear energy is from alpha particles.

43. The method of claim 39, wherein the nuclear energy is from neutrons.

44. The method of claim 43, wherein the neutrons are used for neutron capture therapy.

45. The method of claim 39, wherein the deposited energy is from heavy particles.

46. The method of claim 39, wherein the deposited energy is from a particle beam.

47. The method of claim 1, wherein the deposited energy is absorbed by the magnetic component particles and causes the release of one or more biologically active agents from the particles.

48. The method of claim 1, wherein the deposited energy is photon related.

49. The method of claim 1, wherein the deposited energy causes a beneficial rise or fall in local temperature.

50. The method of claim 1, wherein the deposited energy is ultrasound.

51. The method of claim 1, wherein magnetic component particles further comprises a biologically active agent.

52. A kit for administering radiative therapy, comprising:
b) a unit dose of magnetic component particles;
b) a non-alternating magnet for guiding said particles to a target in the patient once administered to the patient;
c) a source of energy that will deposit energy into the patient once the magnetic component particles have been administered to the patient and magnetically guided to the target;
d) optionally one or more receptacles and instructions for use.

53. The kit of claim 52, wherein the magnetic component particles comprises less than 10% iron oxide.

54. The kit of claim 52, wherein the magnetic component particles comprise a metal with more than 75% metallic iron.

55. The kit of claim 52, wherein the magnetic component particles comprise magnetocarbon particles.

56. The kit of claim 52, wherein the magnetic component particles comprise magnetoceramic particles.

57. The kit of claim 52, wherein the magnetic component particles comprise magneto-polymer magnetic component particles.

58. The kit of claim 52, wherein the magnetic component particles further comprise one or more biologically active agents.

59. The kit of claim 58, wherein the one or more biologically active agents are chosen from the group consisting of antifungals, antineoplastics and antibiotics.

60. The kit of claim 52, also comprising an embolic agent.

61. The kit of claim 52, wherein the source of energy is a RF capacitive heating system.

62. The kit of claim 52, wherein the source of energy is tunable.

63. The kit of claim 52, wherein the source of energy is a source of neutrons.

64. The kit of claim 52, wherein the source of energy is a source of gamma rays.

65. The kit of claim 52, wherein the source of energy is a source of beta particles.

66. The kit of claim 52, wherein the source of energy is a source of alpha particles.

67. The kit of claim 52, wherein the source of energy is a source of heavy particles.

68. The kit of claim 52, wherein the source of energy is a particle beam.

69. The kit of claim 52, wherein the source of energy is a source of electrical energy.

70. The kit of claim 52, wherein the source of energy is a source of alternating magnetic energy.

71. The kit of claim 52, wherein the source of energy is a source of photons.

72. A targetable particle comprising a magnetic component other than metallic iron and either carbon or ceramic material.

73. The targetable particle of claim 72, wherein the particle is a carbon-bearing particle.

74. The targetable particle of claim 73, wherein the carbon is chosen from the group consisting of activated carbon type A, type B, type E, type K, and type KB.

75. The targetable particle of claim 73, wherein the magnetic component is chosen from the group consisting of nickel, cobalt, awaruite, wairauite, pyrrhotite, greigite, troilite, yttrium iron garnet, Alnico 5, Alnico 5 DG, Sm₂Co₁₇, SmCo₅, and NdFeB components.

76. The targetable particle of claim 73, wherein the magnetic component is chosen from the group consisting of nickel, cobalt, awaruite, wairauite, pyrrhotite, greigite, troilite, and yttrium iron garnet components.

77. The targetable particle of claim 74, further comprising one or more biologically active agents.

78. The targetable particle of claim 77 wherein the one or more biologically active agents are chosen from the group consisting of antifungals, antibiotics and antineoplastic agents.

79. The targetable particle of claim 72, wherein the particle is a ceramic-bearing particle.

80. The targetable particle of claim 79, wherein the ceramic material is silica, octadecyl silica or other chemically modified silica, or hydroxyapatite.

81. The targetable particle of claim 79, wherein the magnetic component is chosen from the group consisting of nickel, cobalt, awaruite, wairauite, pyrrhotite, greigite, troilite, yttrium iron garnet, Alnico 5, Alnico 5 DG, Sm₂Co₁₇, SmCo₅, and NdFeB components.

82. The targetable particle of claim 79, wherein the magnetic component is chosen from the group consisting nickel, cobalt, awaruite, wairauite, pyrrhotite, greigite, troilite, and yttrium iron garnet components.

83. The targetable particle of claim 79, further comprising one or more biologically active agents.

84. The targetable particle of claim 83, wherein the one or more biologically active agents are chosen from the group consisting of antifungals, antibiotics and antineoplastic agents.

85. The targetable particle of claim 72, further comprising one or more biologically active agents.

86. The targetable particle of claim 85, wherein the one or more biologically active agents is chosen from the group consisting of antifungal, antibiotic and antineoplastic agents.

87. The targetable particle of claim 72, further comprising one or more excipients.

88. The targetable particle of claim 72, further comprising one or more delivery vehicles.

89. The targetable particle of claim 72 in a unit dose form.
